(19) United States
(12) Patent Application Publication
(10) Pub. No.: US 2010/0216875 A1
(43) Pub. Date: Aug. 26, 2010

(54) METHODS OF TREATING ALZHEIMER'S DISEASE

(76) Inventors: Gregory Hook, La Jolla, CA (US); Vivian Hook, La Jolla, CA (US); Robert Ternansky, San Diego, CA (US)

Correspondence Address: Cooley Godward Kronish LLP, 777 6th Street NW Suite 1100, Washington, DC, 20001, US

(21) Appl. No.: 12/520,375

(22) PCT Filed: Dec. 17, 2007
(86) PCT No.: PCT/US2007/088163

§ 371 Date: Feb. 25, 2010

Related U.S. Application Data
(60) Provisional application No. 60/875,943, filed on Dec. 20, 2006.

(51) Int. Cl. A61K 31/336; A61P 25/28

(52) U.S. Cl. 514/475

(57) ABSTRACT

The invention is directed to a method of treating Alzheimer's disease. The method comprises administering a cysteine protease inhibitor to a patient and thereby affecting the disease. In a preferred method, the cysteine protease inhibitor is a cathepsin B inhibitor. A preferred method causes a reversal in the progression of the Alzheimer's disease, especially an improvement in cognitive deficit. A preferred method also causes a reduction in Aβ. The method includes using a reversible or irreversible cysteine protease inhibitor or a combination thereof.

### CROSS REFERENCE TO RELATED APPLICATIONS

This application claims the benefit of U.S. provisional patent application No. 60/975,943, filed on Dec. 20, 2007, pursuant to 35 U.S.C §119(e)

### STATEMENT OF GOVERNMENT INTEREST

This invention was made with United States Federal government support under grant number 2 R44 AG18044, awarded by the National Institute of Health The Federal government may have certain rights in this invention.

### BACKGROUND OF THE INVENTION

1. Field of the Invention

This invention relates generally to chemistry, medicinal chemistry, enzymology, biochemistry, molecular and cellular biology, genetics, pharmacology, pharmacy, neurobiology, medicine, and drug discovery. In particular, the present invention relates to drugs, pharmaceutical compositions, methods of making drugs, and methods of using drugs, especially for treating and improving Alzheimer's dementia.

2. Brief Description of the State of the Art

Alzheimer's dementia (hereinafter “AD”) is a neuropsychiatric disorder that generally appears in the 6^{th} to 7^{th} decade of life and results in a progressive degeneration of memory and cognitive processes The cognitive deficits severely incapacitate the AD patients to the point where they are totally dependent on family or health care facilities for survival While several palliative drug therapies are available for treating AD, none are effective for any length of time because they do not treat the underlying disease process (9). FDA approved AD drugs include cholinesterase inhibitors, such as doepezil HCl (Aricept™) and rivastigmine tartrate (Exelon™), that cause a temporary and modest improvement but do not stop the progression of the disease. Another drug therapy use a neuroprotective N-methyl-D-aspartate anatagonist, memantine, which provides some temporary benefit in moderate to severe AD patients but also does not stop the progression of the disease.

There are over 4 million Americans and 10 million people world-wide with AD. It is predicted that by the middle of the 21^{st} century, 14 million Americans will have AD. Annual costs in the US and world-wide are $100 billion and $358 billion, respectively (Novartis survey on AD). In light of this growing and expensive problem, a drug that retards or reverses the disease progress is urgently needed

A consistent neuropathological sign of AD is the appearance of extracellular amyloid plaques in cortical and related brain regions. The plaques contain accumulations of Aβ peptides, consisting of Aβ40 and Aβ42 peptides which differ in their carboxy-terminal amino acids. Aβ42 accumulates to a greater extent than Aβ40 in AD patients. Aβ peptides are neurotoxic and are responsible for neuronal loss in brain regions responsible for memory (10-12). Aβ is naturally produced but its normal function is not known. A central hypothesis in the AD field is that the abnormal Aβ accumulation causes neuronal degeneration in brain regions responsible for memory and cognition. Intense research efforts by the academic community and the pharmaceutical industry have focused on developing drug strategies to reduce Aβ levels for the treatment of AD.

The essential role of elevated Aβ in AD was demonstrated in transgenic mice that overexpress a mutant form of APP (β-17). These animals have similar plaques and neuronal lesions in their brains as are found in human AD patients. Moreover, mutant forms of presenilins result in excessive production of Aβ as a result of AD (18, 19). Importantly, a vaccine developed against Aβ42 abolished the neuropathology resulting from overexpression of APP in both young and old animals and improved cognition (20, 21).

Resources are being expended to develop BACE1 inhibitors for treating AD. But despite over 10 years of intense effort, there is no published clinical trial using a BACE1 inhibitor and no peer-reviewed published scientific paper showing that a BACE1 inhibitor effects cognitive function in an animal AD model Only a few peer-reviewed publications show a BACE1 inhibitor affecting brain Aβ in an animal model

### β-Secretase Inhibitors as AD Drugs.

An accepted strategy for reducing abnormal Aβ accumulation is to develop drugs that inhibit Aβ production. Protease inhibitors represent a logical therapeutic approach to prevent proteolytic conversion of the amyloid precursor protein (APP) into the smaller neurotoxic Aβ peptides. Aβ peptides are derived from APP by proteolytic processing (22-24). As shown in FIG. 1, Proteases produce Aβ by cleaving specific amino acid sequences within APP at the N- and C-termini of Aβ. Proteases that convert APP to Aβ have been named “secretases” because Aβ is “secreted.” The proteases that cleave the amino terminal and carboxy-terminal ends of Aβ are called β-secretase and γ-secretase, respectively (FIG. 1). Because β-secretase is required to produce both Aβ40 and Aβ42, drug inhibition of β-secretase is a logical strategy to reduce Aβ peptides in AD. Note that one end of the APP sequence shows the wild-type β-secretase sequence VKM found in the vast majority of AD patients. This site is cleaved by cathepsin B, making this enzyme a likely β-secretase.

### SUMMARY OF THE INVENTION

In one aspect, the invention is directed to a method of treating Alzheimer's disease. The method comprises administering a cysteine protease inhibitor to a patient and thereby affecting the disease In certain aspects the method of the invention may also include use of a cysteine protease inhibitor selected from the group consisting of CA074Me, E64d (loxistatin, EST), Ac-LVK-CHO, Z-VK(Ac)-ald, Z-YS-ald, Z-Q(Trt)S-ald, Z-YV-ald, Z-C(Z)S-ald or Z-C(Z)K(Ac)-ald

The various aspects of the present invention may cause a reduction in Aβ and/or a reversal in the progression of Alzheimer's disease in the patient.

### BRIEF DESCRIPTION OF THE DRAWINGS

FIG. 1 shows that proteases produce Aβ by cleaving specific amino acid sequences within APP at the N- and C-termini of Aβ

FIG. 2a shows that the Z-Val-Lys-Met-MCA cleaving activity of cathepsin B purified from secretory vesicles was completely inhibited by the cysteine protease inhibitor E64c (10 μM) and by CA074 (1 μM), a selective inhibitor of cathepsin B

FIG. 2b shows that the endogenous β-secretase activity in regulated secretory vesicles (isolated), monitored with Z-Val-Lys-Met-MCA, was completed inhibited by the cysteine protease inhibitor E64c (10 μM) and by CA074 (1 μM)

FIG. 3 shows that irreversible cathepsin B inhibitors block Aβ production from endogenous APP in isolated regulated secretory vesicles.

FIG. 4 shows that Aβ is mostly secreted via the regulated secretory pathway and that CA074Me (+) selectively reduces Aβ secreted from that pathway

FIG. 5a shows a 30% or 50% reduction, after 7 or 30 days treatment with E64d (+), in the total Aβ as measured in brain extracts by ELISA assays

FIG. 5b shows that treatment resulted in a 27% or 50% reduction, after 7 or 30 days treatment with E64d (+), in the total synaptosomal Aβ as measured in isolated synaptosomes by ELISA assays

FIG. 6a shows a 50% or 60% reduction, after 7 and 30 days treatment with CA074Me (+), in Aβ as measured in brain extracts by ELISA assays.

FIG. 6b shows a 45% or 50% reduction, after 7 or 30 days treatment with CA074Me (+), in total Aβ as measured in isolated synaptosomes by ELISA assays

FIG. 6c shows a 50% reduction of both Aβ40 and Aβ42 in brain extracts after treatment for 30 days treatment with CA074Me (+), as measured using specific ELISA assays.

FIG. 7 shows that CA074Me reduces β-secretase activity, as measured by the APP-derived CTFR fragment.

FIG. 8a shows measurements of Aβ40 and Aβ42 in transgenic mouse (APPIn) brains by ELISA assays and expressed as ng Aβ/g (brain weight), mean±s.e.m. after treatment with cysteine protease inhibitors.

FIG. 8b, shows a quantitative assessment of amyloid load with or without treatment with cysteine protease inhibitors by using Aβ immuno-staining, expressed as percent amyloid in brain tissue (cortex examined). The treatment was found to significantly reduce amyloid load

FIG. 8c shows that treatment of APPIn mice resulted in a significantly lower average latency period (seconds) in animals that were tested for cognitive function by the Morris water maze test

FIG. 9a shows that total Aβ peptide levels as measured by ELISA and expressed as nanograms Aβ (Aβ40+Aβ42) per gram brain tissue (n=3) were significantly increased in the APPIn mice.

FIG. 9b shows an amyloid load in APPIn mice but not in wild-type mice, as determined from mouse brain cortex sections stained for amyloid deposits using anti-Aβ.

FIG. 9c shows that the latency period for untreated APPIn mice was 2.3 times longer than for wild-type mice when both were subjected to Morris-water maze tests

FIG. 10 shows that icy infusion of Ac-LVK-CHO into gp brains at 1 mg/ml, 2.5 μl/hr (0.15 mg/kg/day) for 30 days in a 1.5% DMSO/saline carrier solution using mini-pumps significantly reduced both Aβ40 and Aβ42 by about 70% and 60%, respectively, relative to controls

FIG. 11 shows the CTFβ levels as a percent of control for the treated and control groups in FIG. 10. The treatment resulted in a about a 65% reduction in CTFβ.

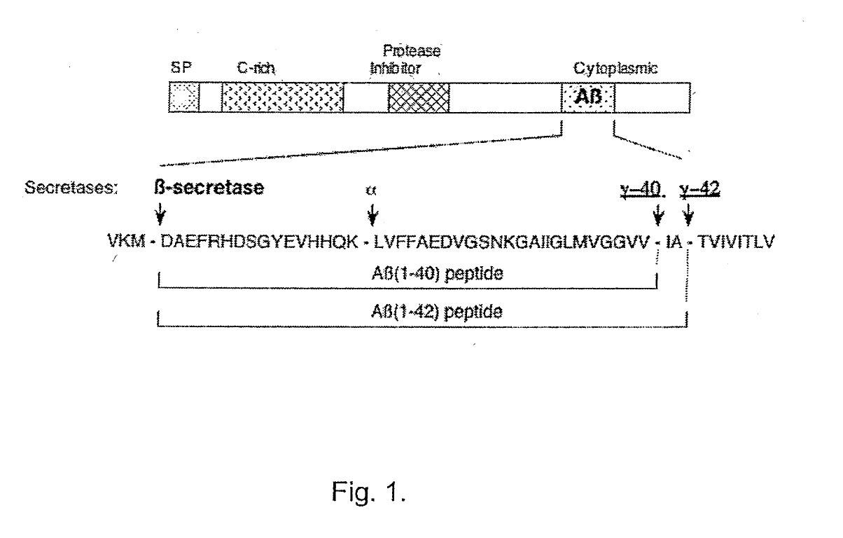

### DETAILED DESCRIPTION OF THE PREFERRED EMBODIMENTS

In one aspect, the invention is directed to a method of treating Alzheimer's disease. The method comprises administering a cysteine protease inhibitor to a patient and thereby affecting the disease. In a preferred method, the cysteine protease inhibitor is a cathepsin B inhibitor and more preferably where the inhibitor is E64d. A preferred method causes a reversal in the progression of the Alzheimer's disease, especially an improvement in cognitive deficit. A preferred method also causes a reduction in Aβ. The method includes using cysteine protease inhibitor that is an irreversible inhibitor or a reversible inhibitor, preferably a cathepsin B irreversible or reversible inhibitor.

As used herein, the term “cognitive” includes “spatial memory” and terms such as “cognitive deficit” or “cognitively deficient” and such include “spatial memory deficit” or “spatial memory deficient”.

In certain aspects the method of the invention may also include use of a cysteine protease inhibitor selected from the group consisting of CA074Me, E64d (loxistatin, EST), Ac-LVK-CHO, Z-VK(Ac)-ald, Z-YS-ald, Z-Q(Trt)S-ald, Z-YV-ald, Z-C(Z)S-ald or Z-C(Z)K(Ac)-ald. The various aspects of the present invention may cause a reduction in Aβ and/or a reversal in the progression of Alzheimer's disease in the patient.

Suitable amounts and routes of administration for these cysteine protease inhibitors for administration to achieve the desired effects can be determined by a skilled person using the test methodologies described in detail below. The effects of administration of these cysteine protease inhibitors on cognitive deficit and levels of Aβ can also be determined using the test methods described in detail below.

### Cathepsin B is a β-Secretase in the Regulated Secretory Pathway

The key to developing effective secretase inhibitors is to identify the proteases that produce Aβ. To this end, experience in the proteolytic processing of neuropeptides was applied (25-37). Neuronal cells have a regulated secretory pathway through which neuropeptides are produced and secreted in response to a triggering event (38-41). Neurons, like all cells, also have a constitutive secretory pathway through which material is secreted independently of a stimulus. The regulated secretory pathway contains different proteases than in the constitutive secretory pathway (42-45). Neurons secrete most of the extracellular Aβ via the regulated secretory pathway (46-49).

Criteria for identifying a protease that produces a neuropeptide in the regulated secretory pathway have been established (27, 35-41). First, the processing protease must be present in the regulated secretory pathway of the neuron. Second, the protease must have the cleavage specificity to produce the neuropeptide. Third, inhibition of the protease must reduce production of the neuropeptide. Application of these criteria resulted in identification of the cysteine protease, cathepsin B, as a candidate (β-secretase in the regulated secretory pathway of neurons (50, 51). Others, looking at the constitutive secretory pathway, identified the aspartyl protease, BACE1, as a (β-secretase (52-56), which is discussed and compared below.

Primary bovine chromaffin cells contain the human brain APP isoform and secrete APP in response to cholinergic agonists (58-60). Chromaffin cells naturally produce large amounts of Aβ via the regulated secretory pathway and purified regulated secretory vesicles from these cells contain APP, Aβ, β- and γ-secretases 1.5 and presenilin (50). Using a peptide substrate that mimics the wild-type (β-secretase cleavage site of human APP, (β-secretase activity was purified 250,000 fold from purified regulated secretory vesicles. Using a selective affinity label, a 31 kDa protein band was observed, and its (β-secretase activity was found to be inhibited by the cathepsin B specific inhibitor, CA074. The band was identified as cathepsin B by tryptic digestion and peptide microsequencing using tandem mass spectrometry. Immunoelectron microscopy found that cathepsin B co-localized with Aβ in the regulated secretory vesicles. Further, as shown below, cysteine protease inhibitors, and in particular cathepsin B-specific inhibitors, reduced β-secretase activity and Aβ production in the isolated regulated secretory vesicles. The cell permeable cathepsin B inhibitor, CA074Me, reduced CTFβ production (a measure of (β-secretase activity) and reduced production and secretion of Aβ in the regulated secretory pathway of primary chromaffin cell cultures (51) As shown in Table 1 below, cathepsin B was found to readily cleave the peptide substrate, Z-Val-Lys-Met-MCA, which mimics the wild-type β-secretase cleavage site of human APP found in 99% of AD patients But cathepsin B did not cleave the peptide substrate, Z-Val-Asn-Leu-MCA, which mimics a mutated (β-secretase cleavage site of human APP called the Swedish mutation (APPswe) found in only one family (50). Moreover, as shown below, cathepsin B inhibitors selectively inhibit the production and secretion of Aβ by the regulated secretory pathway of neuronal cells.

Fluorescent In Vitro Assays for Screening Compounds that Inhibit Wild-Type β-Secretase Activity

A high throughput screening (HTS) assay was developed to measure protease activity that cleaves the wild-type β-secretase cleavage site. The substrate Z-Val-Lys-Met-MCA mimics the wild-type β-secretase cleavage site upon cleavage between Met-MCA, which generates fluorescent MCA. The free MCA fluorescence provides sensitive and quantitative measurement of protease activity. This assay provides a rapid (30 minutes) and sensitive assay of activity for cleaving the wild-type β-secretase cleavage site of APP, expressed in majority of the AD population Specifically, the Z-Val-Lys-Met-MCA cleaving assay can be used to detect endogenous β-secretase activity in regulated secretory vesicles.

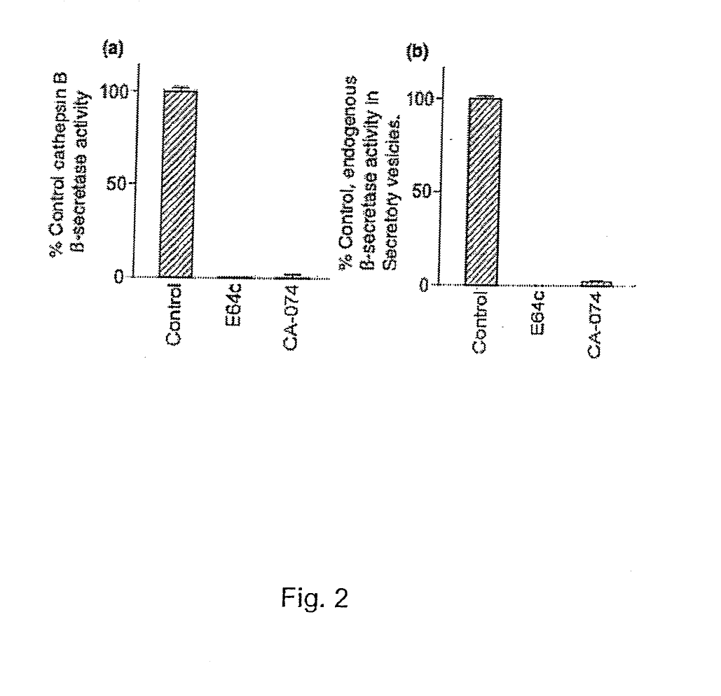

FIG. 2 shows that E64c, the active form of the prodrugE64d (loxistatin, EST) and CA074 inhibit endogenous (β-secretase cleavage in isolated regulated secretory vesicles. As shown in FIG. 2a, the Z-Val-Lys-Met-MCA cleaving activity of cathepsin B purified from secretory vesicles was completely inhibited by the cysteine protease inhibitor E64c (10 μM) and by CA074 (1 μM), a selective inhibitor of cathepsin B. As shown in FIG. 2b, the endogenous β-secretase activity in regulated secretory vesicles (isolated), monitored with Z-Val-Lys-Met-MCA, was completed inhibited by the cysteine protease inhibitor E64c (10 μM) and by CA074 (1 μM).

### Cathepsin B Cleaves the β-Secretase Site in APPwt and not APPswe

A comparison of cathepsin B cleavage of the β-secretase site in APPwt and APPswe using peptidomimetics, Z-V-K-M-MCA and Z-V-N-L-MCA, respectively, was conducted. As shown in Table 1, cathepsin B cleaves the wild-type β-secretase site, rather than the Swedish mutant site

TABLE 1
Cathepsin B Preference for Cleavage of the Wild-Type β-Secretase
Site

Relative β-Secretase Activity pmol
MCA/min/μg cat B

| Protease | Wild Type Site | Swedish Mutant Site |
|---|---|---|
| Cathepsin B | 547 | 0.2 |

### In Vivo Confirmation of the Cathepsin B Cleavage Preference

The ability of cathepsin B to cleave the β-secretase site in APPwt is confirmed in vivo. As discussed above, CA074Me reduces guinea pig (gp) brain CTFβ, which is a measure of (β-secretase activity, and gpAPP contains the wild-type β-secretase site. Thus, cathepsin B inhibition in vivo reduces wild-type β-secretase activity

Cathepsin B's inability to cleave the β-secretase site in APPswe was also confirmed in viva. Knocking out the gene encoding cathepsin B in transgenic mice expressing APPswe has no effect on brain CTFβ (57) and confirms that cathepsin B has no in vivo β-secretase activity for the mutant site.

### Cathepsin B Inhibitors Block Aβ Production in Isolated Bovine Regulated Secretory Vesicles

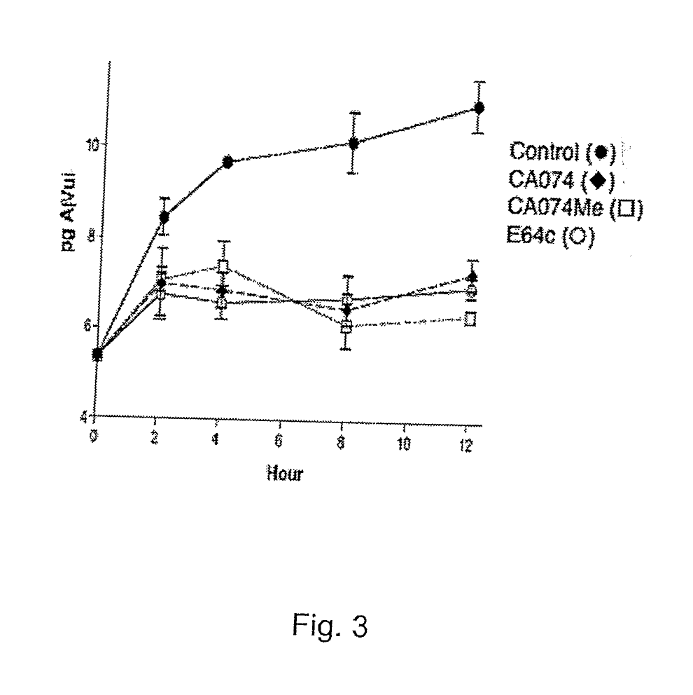

Regulated secretory vesicles isolated from chromaffin cells naturally produce Aβ from endogenous APP when incubated at 37° C. in vitro for several hours. These vesicles can be used as an in vitro screen for compounds that inhibit Aβ production. As shown in FIG. 3, irreversible cathepsin B inhibitors block Aβ production from endogenous APP in isolated regulated secretory vesicles. The effects of cathepsin B inhibitors, CA074 (10 μM, ♦) and CA074Me (10 μM, □), on the production of Aβ40 from endogenous APP in regulated secretory vesicles isolated from chromaffin cells was evaluated in time course studies. Cysteine protease inhibitor E64c (10 μM, ◯) and Controls without inhibitors () were included. Each inhibitor was tested in triplicate; values represent x±sem (*statistically significant with p<0.05). Thus, the in vitro regulated secretory vesicle assay can screen for compounds that inhibit Aβ production and it was found that cathepsin B inhibitors are effective at blocking Aβ production.

### CA074Me Reduces Regulated Aβ Secretion by Primary Neuronal Cell Cultures

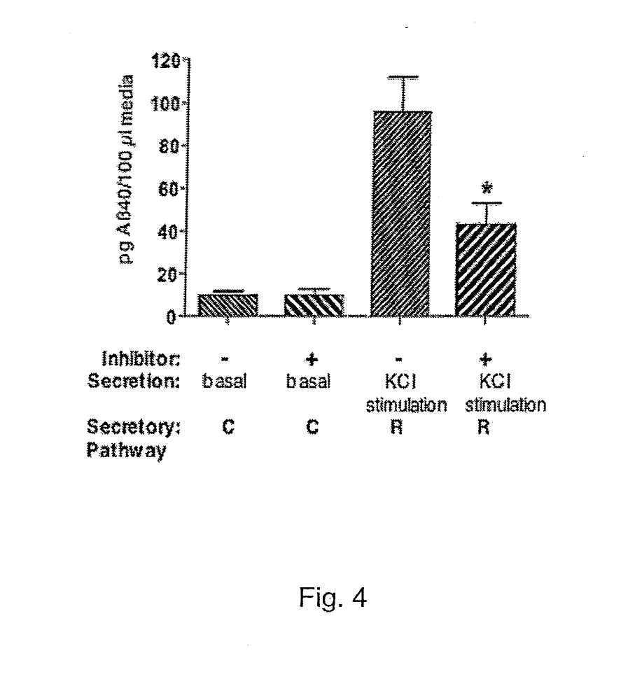

Neuronal bovine chromaffin cells in primary culture can be used to select compounds that affect Aβ in the regulated or constitutive pathway. Regulated secretory Aβ is determined by measuring Aβ in the cell culture media after inducing the cells to secrete using a triggering stimulus, such as KCl. The constitutive secretory Aβ is determined by measuring Aβ in cell culture media (also called “conditioned media”) from resting cells. As shown in FIG. 4, Aβ is mostly secreted via the regulated secretory pathway and CA074Me selectively reduces Aβ secreted from that pathway. FIG. 4 shows the Aβ40 in culture media due to constitutive (C) and regulated (R) secretion by primary neuronal chromaffin cells in culture. Regulated secretion was induced by exposing the cells to KCl (50 mM) for 15 minutes. Comparing untreated cells (−) shows that significantly more Aβ40 is produced by regulated secretion (R) than by constitutive secretion (C). Treated cells (+) received CA074Me (50 μM) for 18 hours. The Aβ40 produced by regulated secretion of the treated cells is about 50% less than that produced from regulated secretion by untreated cells. In contrast, the Aβ40 produced by constitutive secretion from treated cells is the same as the constitutive secretion from untreated cells 0.5 Results are x±sem. *Statistically significant (p<0.05). These results demonstrate that: (1) most Aβ is produced via the regulated secretory pathway, and (2) a cathepsin B-specific inhibitor selectively reduces Aβ production and secretion in that pathway.

Cathepsin B is present in the regulated secretory vesicles where Aβ processing occurs. Cathepsin B cleaves the wild-type 6-secretase cleavage site of human APP, but not the mutated form. Cathepsin B inhibitors, E64d and CA074Me, reduce 6-secretase activity in isolated regulated secretory vesicles and in the regulated secretory pathway of chromaffin cells. Moreover, those inhibitors also reduce Aβ production in those vesicles and cells. As such, cathepsin B satisfies the criteria above and is a candidate β-secretase of the regulated secretory pathway.

Irreversible Cathepsin B Inhibitors Reduce Brain and Synaptosome Aβ and CTFβ in the Guinea Pig, which is a Natural Animal Model of Human APP Processing

The effects of cathepsin B inhibitors on brain and synaptosome Aβ and CTFβ in the non-transgenic guinea pig (gp) model of human APP processing were studied GpAPP contains the wild-type β-secretase site, gpAβ is identical to human Aβ and gpAβ is produced in sufficient quantity that a reduction can be readily determined. Thus, the gp model is an excellent natural model of human 6-secretase APP processing (5-7).

As shown below, icy administration of the cathepsin B specific inhibitor, CA074Me, to gp at a dose of 0.15 mg/kg/day reduced both Aβ40 and Aβ42 in the brain by 50-60%. Moreover, this treatment also reduced by about the same amount the Aβ in synaptosome preparations, which are enriched with synapses, and are a measure of regulated secretion. Further, this treatment also similarly reduced brain CTFR, which is a measure of β-secretase activity. In addition, the general cysteine protease inhibitor, E64d, at the same dose and route of administration, also reduced brain Aβ by 50%. The conclusion is that cathepsin B inhibitors significantly reduce brain Aβ produced by the regulated secretory pathway because cathepsin B inhibitor treatments caused a large reduction in both brain and synaptosome Aβ. A corollary conclusion is that cathepsin B is a likely brain β-secretase because cathepsin B inhibitors also reduced brain β-secretase activity as measured by CTFβ.

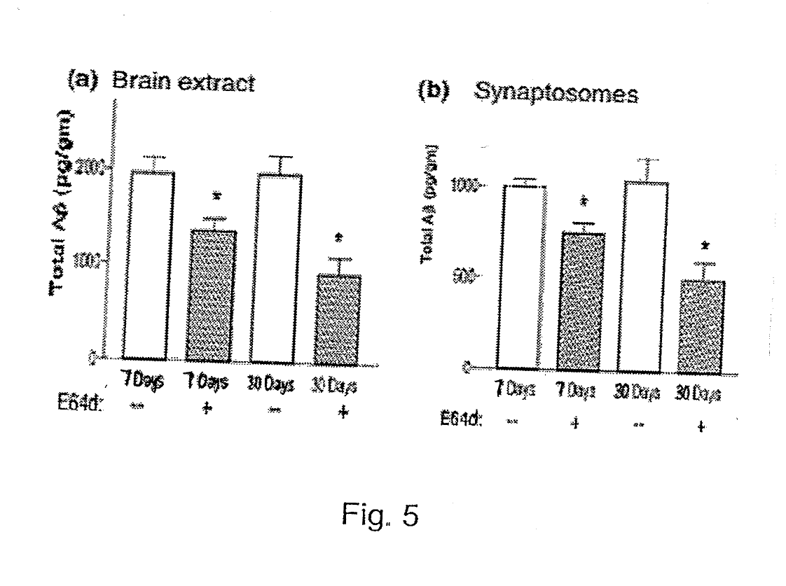

Guinea pig (gp) is a natural, non-transgenic model of human APP processing gpAPP is 98% homologous to human APP, contains the human wild-type β-secretase site, and gpAβ is identical to human Aβ. Brain synaptosome preparations are enriched with neuronal synapses which are the sites at which regulated secretion occurs and are an in vivo measure of regulated secretory secretion CTFβ is an APP fragment that results from p-secretase cleavage of APP and reflects in vivo β-secretase activity. As shown in FIG. 5, E64d (loxistatin) reduces gp brain and synaptosomal Aβ in vivo. E64d was icy administered into gp brains (0.15 mg/kg/day) for 7 or 30 days. In FIG. 5a, total Aβ was measured in brain extracts by ELISA assays and treatment resulted in a 30% or 50% reduction after 7 or 30 days, respectively. In FIG. 5b, total synaptosomal Aβ is shown. Total Aβ was measured in isolated synaptosomes by ELISA assays and treatment resulted in a 27% or 50% reduction after 7 or 30 days, respectively

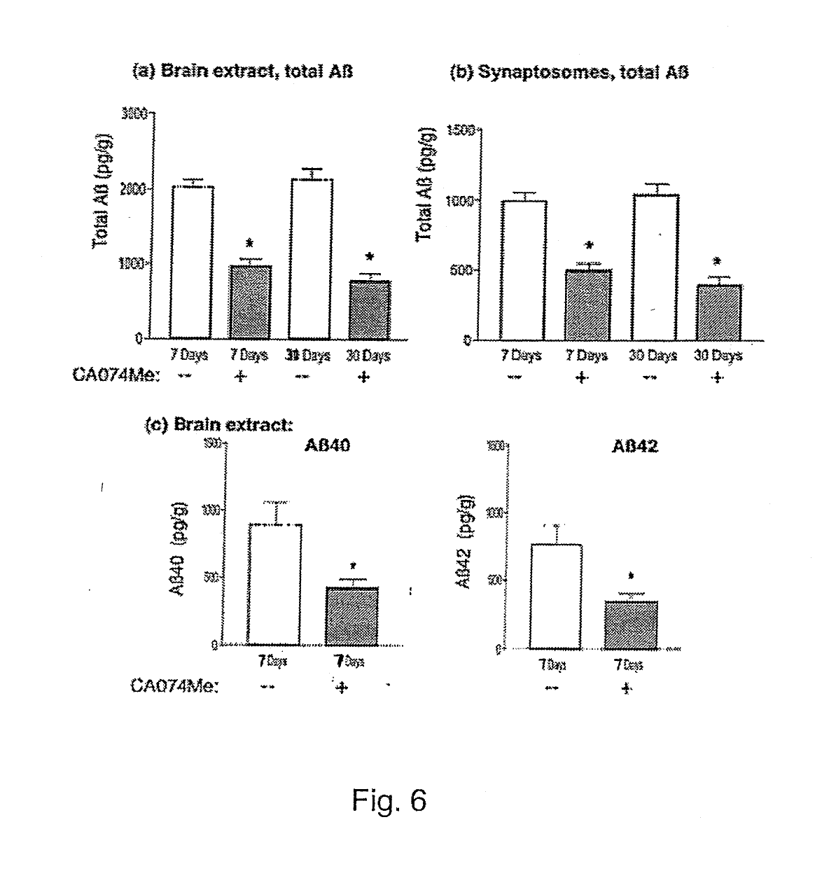

As shown in FIG. 6, CA074Me reduces gp brain Aβ, synaptosomal Aβ and Aβ40 and Aβ42 equally. CA074Me (0.15 mg/kg/day) was icy infused into gp brains with mini pumps. In FIG. 6a, Aβ was measured in brain extracts by ELISA assays and treatment resulted in 50% or 60% reduction after 7 and 30 days, respectively. In FIG. 6b, total Aβ was measured in isolated synaptosomes by ELISA assays and treatment resulted in a 45% or 50% reduction after 7 or 30 days, respectively. In FIG. 6c, specific ELISA assays were used to measure Aβ40 and Aβ42 separately in brain extracts and treatment for 7 days resulted in a 50% reduction of both. An equal reduction of Aβ40 and Aβ42 is consistent with the inhibitor affecting p-secretase activity

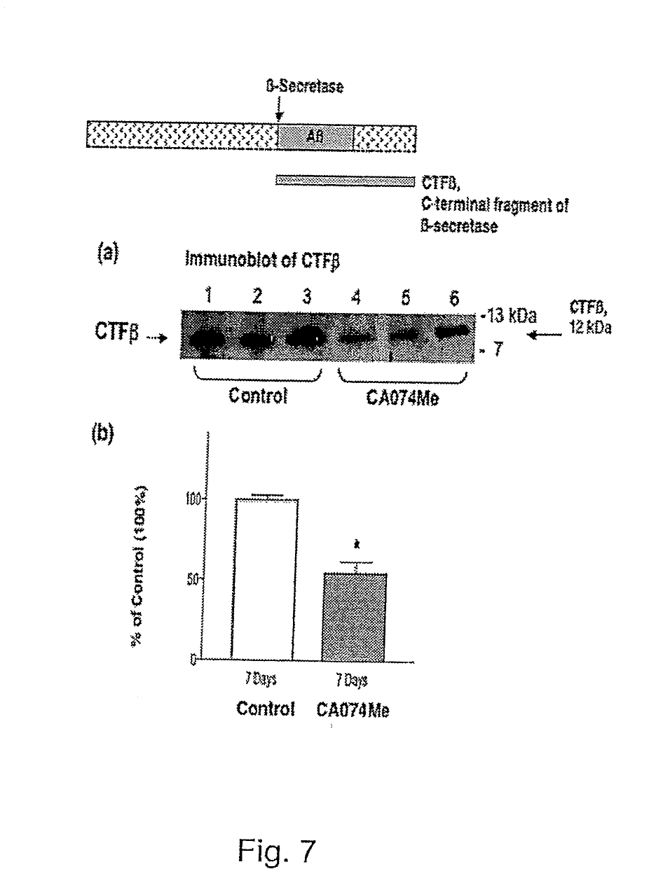

To better evaluate the effects of CA074Me on β-secretase activity, relative brain levels of CTRβ were assessed by western blots. The CTRβ fragment results from processing of APP at the β-secretase site, which produces the C-terminal β-secretase fragment (CTFβ) of 12 kDA on SDS-PAGE gels. As shown in FIG. 7, CA074Me reduces (β-secretase activity as measured by the APP-derived CTFβ fragment. CA074Me (0.15 mg/kg/day) was infused into guinea pig brains for 7 days. Brain tissue was extracted for western blot analyses of CTFβ (12 kDa) detected with anti-Aβ (FIG. 6a). Replicate samples of control (lanes 1-3) and CA074Me treated (lanes 4-6) animals showed a 50% decrease in the amount of CTFβ in animals given CA074Me. Thus, irreversible cathepsin B inhibitors reduce brain Aβ in the regulated secretory pathway and (β-secretase activity in vivo,

Cathepsin B Correlated in Humans with AD

Cathepsin B is upregulated in the brain of an AD patient, and is colocalized with Aβ in amyloid plaques (61,62). Cathepsin B is elevated in cerebrospinal fluid (CSF) of AD patients (63), which is consistent with its regulated secretion from neurons. Age-related changes in expression of brain cathepsin B may be involved in the late onset of the disease (64,65). These findings by others are consistent with the present results showing that cathepsin B produces secreted brain Aβ

### Cathepsin B Inhibition is not Toxic

Cathepsin B is ubiquitously distributed throughout the body and is involved in a wide range of functions. Originally identified as a digestive lysosomal protease, cathepsin B also has other specific functions, such as MHC antigen mediated presentation. As such, cathepsin B inhibition could raise toxicity concerns. However, the data show that inhibiting cathepsin B does not result in toxic reactions.

### Cathepsin B Knockout Mice are Normal Mice

A review of cathepsin B knockout mice found that the knockout animals cannot be distinguished from their wild-type counterparts, are fertile and do not exhibit behavior deficits (66) Extensive microscopic and histological examination of multiple tissues did not reveal any alterations and demonstrated that cathepsin B is dispensable for MHC mediated antigen presentation.

### Cysteine Protease Inhibitors Safe in Clinical Trials and Animal Toxicity Studies

The general cysteine protease inhibitor, E64d, was tested in Japan for treating muscular dystrophy in Phase I through III trials, but was discontinued due to a lack of efficacy (2, 3, 67) in that indication. Nonetheless, in the Phase I study, healthy, young (25 years old) males given 5 mg/kg/day oral doses of E64d had no change in subjective or objective symptoms or clinical tests. No change in pulse, resting blood pressure, body temperature or grip force was observed and no change was found in the general hematological tests, blood chemistry tests or urinalysis. Pharmacokinetics showed good absorption, but no accumulation occurred and about 30% of the total dose was collected in urine.

E64d oxicity has been tested in mice, rats and dogs (68). Those studies showed that E64d has an excellent therapeutic window with an LD₅₀ in mice, rats and dogs

### BACE1 β-Secretase Inhibitors

The approach used to identify the aspartyl protease, BACE1, as a β-secretase established that another entirely different protease from cathepsin B, BACE1, produces Aβ in the basal or constitutive secretory pathway and cleaves the Swedish mutant β-secretase site. The elucidation of BACE1 as a β-secretase is explained below.

### BACE 1 β-Secretase Activity Identified in the Constitutive Secretory Pathway

Many years ago, BACE1 was identified as a β-secretase in HEK293 and other cell lines that produce Aβ in “conditioned media” (52-56). The Aβ in the “conditioned media” was that produced by the cells under basal or resting conditions and thus represents the Aβ produced by the constitutive secretory pathway. Based on the cell biology of secretory pathways (44, 45), identifying Aβ in the “conditioned media” establishes that BACE1 is a β-secretase in the constitutive secretory pathway.

### BACE1 Different Amino Acid Residues at the Swedish Mutant β-Secretase Site Compared to Wild-Type Site Suggests Different Proteases

BACE1 was also identified using transgenic cells that express human APPswe in which the β-secretase site contains two amino acid mutations that make this β-secretase site very different from that found in human APPwt. Nonetheless, several studies believed it appropriate to utilize the mutant β-secretase site to identify a protease with a preference for processing the mutant form because those mutations lead to increased Aβ production (52-56). Using this mutant form, BACE1 was identified as a β-secretase of APPswe and readily cleaves the mutant form BACE1 was later found to have extremely poor ability to cleave the wild-type β-secretase site (72).

Proteases are known to possess selectivity for cleavage at amino acids having different chemical properties. Therefore, because the charged Lys residue at the wild-type β-secretase site (-Lys-Met-Asp-) differs chemically from the neutral Asn residue of the mutant site (-Asn-Leu-Asp), different proteases may recognize and cleave the wild-type site compared to the Swedish mutant site. Cathepsin B prefers to cleave the wild-type β-secretase site whereas BACE 1 prefers the Swedish mutant site. Thus, use of different cleavage site specificities for elucidation of proteases led to identification of two different proteases—cathepsin B and BACE 1.

in vivo Reduction of BACE 1 by Genetic Knockout Approaches

Data from studies of BACE1 knockout mice are consistent with a role for BACE 1 in the constitutive secretory pathway, with preference for the Swedish mutant β-secretase site Homogenized brain from BACE1 knock out animals detected no β-secretase activity and concluded therefore that BACE1 is the only β-secretase. However, the assay conditions used to detect (β-secretase activity contained the cysteine protease inhibitor E64c which prevented detection of cysteine protease p-secretase activity such as that due to cathepsin B (73). A reducing agent is essential for cathepsin B activity and its absence in β-secretase assays from BACE1 knockout mice (73) also precluded detection of cathepsin B.

Resting neuronal cultures from BACE1 knockout mouse brains showed reduced basal secretion of Aβ into conditioned media (74, 75), indicating a role for BACE 1 for Aβ production in the constitutive secretory pathway Another study examined the absence of BACE 1 in mice that over-expressed the Swedish mutant form of APP (76, 77). Based on the preference of BACE1 for cleaving the Swedish mutant β-secretase site, it is logical that the absence of BACE1 resulted in reduced cleavage of Swedish mutant APP. These studies have not assessed the effect of BACE1 knockout on production of Aβ in the regulated secretory pathway.

### In Vivo Reduction of BACE 1 by Chemical Inhibitor Approaches

BACE 1 inhibitors have been shown to reduce brain Aβ by about 30% (78, 79). For example, the BACE 1 inhibitor KMI-429 (76) caused a maximum reduction in brain Aβ of about 30-40% in wild-type mice using a high icy dose of 200 mg/kg/day.

### Cathepsin B and BACE 1, Participate in AβProduction Via Processing of APP

The data support the hypothesis that production of cellular Aβ by β-secretase utilizes two parallel protease pathways consisting of cathepsin B for Aβ biosynthesis in the regulated secretory pathway of neurons, and BACE 1 for Aβ production in the constitutive secretory pathway of such cells

Cathepsin B Inhibitors Reduce Cognitive Deficit, Plaque Pathology and Brain Aβ in the Transgenic APPIn Mouse, which Mimics AD Pathology and Behavior.

To evaluate efficacy, the cathepsin B inhibitors were studied in a transgenic AD mouse model. Mice that express human APP having the London mutation were developed that overproduce brain Aβ, generate brain plaques and have reduced cognitive ability (8). Importantly, the London mutant APP (APPIn) produced in these transgenic animals has the wild-type β-secretase cleavage site and a mutated γ-secretase site, which causes the Aβ overproduction. Thus, this is an excellent model for evaluating β-secretase inhibitors because the β-secretase activity in these animals models is comparable to that found in the vast majority of AD patients.

The results of these studies are shown below. Briefly, icy administration of 0.015 mg/kg/day of CA074Me or E64d to APPIn mice caused a significant 50% decrease in brain Aβ40 and Aβ42 and a similar reduction of brain plaque load. More importantly, the cathepsin B inhibitors caused an 80% improvement in the cognitive deficit in these animals. The primary and significant conclusion that can be drawn from these data is that cathepsin B inhibitors are AD therapeutics because they are efficacious in an AD animal model. More importantly, the data show that cathepsin B inhibitors can reverse the cognitive deficit caused by AD.

### Irreversible Cathepsin B Inhibitors Improve Cognitive Ability and Reduce Brain Aβ and Plaque Load in a Transgenic AD Animal Model

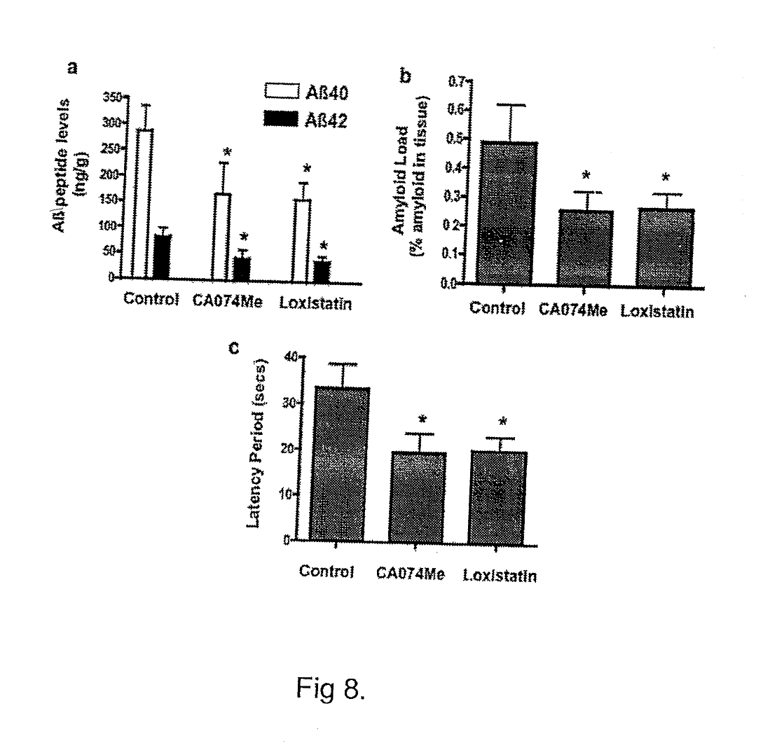

Transgenic mice expressing human APP containing the London mutation (APPIn) mimic AD function and pathology in that they are cognitively deficient, have brain amyloid plaque and elevated Aβ (8). APPIn has the wild-type β-secretase site, which is found in 99% of AD patients and is the site cleaved by cathepsin B. As shown in FIG. 8, CA074Me or E64d treatment of 8 month old APPIn mice reduced brain Aβ and amyloid plaque and improved cognitive function. CA074Me or E64d were administered by icy infusion in brains (0.015 mg/kg/day) for 28 days (8 animals per treatment group). As shown in FIG. 8a, Aβ40 and Aβ42 were measured by ELISA assays and expressed as ng Aβ/g (brain weight), mean±s.e.m. As shown in FIG. 8b, amyloid load was quantitatively assessed by Aβ immuno-staining, and is expressed as percent amyloid in brain tissue (cortex examined). The treatment was found to significantly reduce amyloid load. As shown in FIG. 8c, animals were tested for cognitive function by the Morris water maze test and treatment of APPIn mice resulted in a significantly lower average latency period (seconds). Moreover, these results were statistically significant with p<0.005 for each treatment group compared to the relevant control.

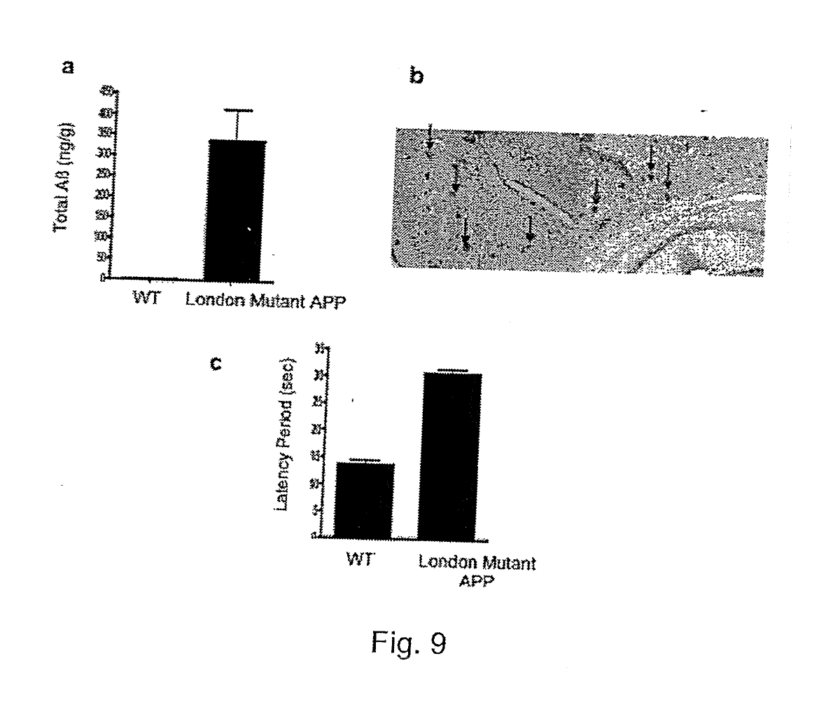

As shown in FIG. 9, APPIn mice show elevated Aβ, amyloid deposits, and cognitive deficit as compared to wild-type mice London mutant APP mice were evaluated at 8 months of age to confirm high levels of brain All, amyloid deposits, and the presence of cognitive deficit. As shown in FIG. 9a, total Aβ peptide levels were measured by ELISA and expressed as ng Aβ (Aβ40+Aβ42) per gram brain tissue (n=3) and found to be significantly increased in the APPIn mice. As shown in FIG. 9b, mouse brain cortex sections stained for amyloid deposits using anti-Aβ showed an amyloid load in APPIn mice but not in wild-type mice. As shown in FIG. 9c, wild-type and APPIn mice subjected to Morris-water maze tests as described above showed that the latency period for untreated APPIn animals was 2.3 times longer than for wild-type animals. Comparing FIG. 8c reveals that treating the APPIn animals dropped the latency period to only 1.2 times that of wild type animals (n=3). Thus, irreversible cathepsin B inhibitors are efficacious in this AD animal model, causing an 80% improvement in cognitive function and a 50% decrease in amyloid plaque load and brain Aβ. Thus, these tests indicate that Cathepsin B inhibitors are effective at reversing the cognitive deficit caused by AD

Cathepsin B Inhibitors have No Effect on a Transgenic APPIn Mouse Expressing Human APP Containing the Swedish Mutant β-Secretase Site.

Cathepsin B inhibitors were also evaluated in another transgenic mouse that expressed human APP containing both the Swedish mutant β-secretase and the London γ-secretase site (APPswe/In). The same treatment procedure as used for the APPIn mice resulted in no significant change in memory, amyloid plaque load, Aβ40, Aβ42, CTFβ, or sAPPα I the APPswe/In mice (Table 2)

The fundamental difference between the APPswe/In mice compared to the APPIn mice is the expression of the Swe mutant β-secretase site in the APPswe/In mice rather than the wt β-secretase site expressed in the APPIn. In particular, the substitution of the neutral asparagine residue in the Swe β-secretase site for the positively charged lysine found in the wt β-secretase site changes the substrate structure, which alters the biochemical properties of β-secretase cleavage.

The lack of effectiveness of cathepsin B inhibitors in mice expressing the Swe β-secretase site was expected based on the cathepsin B assay used to select the inhibitors. Cathepsin B showed poor cleavage efficiency for the Swe β-secretase site, but showed high selectivity for cleaving the wt β-secretase site (Table 1). Thus, cathepsin B inhibitors were expected to be efficacious in animals expressing the wt β-secretase site but not the swe β-secretase site.

In contrast to the APPswe/In mice, which showed no response to the cathepsin B inhibitors, cathepsin B inhibitors were efficacious in APPIn mice expressing the wt β-secretase site for improvement in memory deficit with reductions in amyloid plaque load and Aβ peptides.

There was no significant difference in the mean latency period or mean distance traveled for the untreated controls in both the APPswe/In and APPIn groups of mice. However, a 2.3-fold greater amyloid plaque load was observed in the control APPswe/In mice relative to the control APPIn mice.

TABLE 2
Cysteine Protease Inhibitors Have No Effect in APPswe/In Mice.

| | Mean + % SEM | | | Significance |
|---|---|---|---|---|
| Parameter | Control | CA074Me | Loxistatin | (p < 0.05) |
| Latency | 38 + 8% | 37 + 12% | 40 + 10% | ns¹ |
| Period (sec.) | | | | |
| Distance | 253 + 10% | 246 + 13% | 270 + 12% | ns |
| Traveled (cm.) | | | | |
| Amyloid load | 1.14 + 4% | 1.07 + 4% | 1.06 + 4% | ns |
| (% area) | | | | |
| Aβ40 | 103 + 5% | 91 + 4% | 92 + 7% | ns |
| (% control) | | | | |
| Aβ42 | 100 + 4% | 97 + 3% | 101 + 3% | ns |
| (% control) | | | | |
| CTFβ | 96 + 4% | 97 + 3% | 100 + 3% | ns |
| (% control) | | | | |
| sAPPα | 105 + 5% | 101 + 4% | 98 + 3% | ns |
| (% control) | | | | |

¹ns = means not significantly different by ANOVA.

### A Reversible Cathepsin B Inhibitor

E64d and CA074Me both contain a core epoxysuccinyl structure (69-71). These compounds inhibit proteases by a covalently bonding via their epoxy ring to the active site of the protease. As such, these compounds chemically react and irreversibly inhibit the protease.

Although irreversible inhibitors are used as pharmaceuticals, they are generally not preferred because the reactive nature of these compounds raises issues of potential toxicity due to nonspecific reactions. These concerns are increased when the irreversible inhibitor is chronically administered and when the patient population is frail. Chronic administration and frail patients are both typical for administration of AD drugs. Thus, reversible AD inhibitors may have advantages because nonspecific reactions are less likely to occur and thus toxicity concerns may be reduced relative to the use of irreversible inhibitors, although, as stated above, E64d has been shown to be non-toxic in human clinical trails.

### Reversible Cathepsin B Inhibitor Reduces Brain Aβ and CTFβ in the gp.

The known reversible cathepsin B inhibitor, acetyl-L-leucyl-L-valyl-L-lysinal (Ac-LVK-CHO), is effective at reducing brain Aβ and β-secretase activity in the guinea pig animal model. This peptidomimetic is cell permeable and has an IC₅₀ of 4 nM for cathepsin B inhibition (4). As shown below, icy administration of Ac-LVK-CHO for 30 days at 1 mg/ml, 2.5 μl/hr (0.15 mg/kg/day) significantly reduces brain Aβ40 and Aβ42 by about 70% and 60%, respectively, relative to controls. The Ac-LVK-CHO also reduces brain CTFβ by about 65% relative to controls. These results are approximately the same as for the irreversible cathepsin B inhibitor, CA074Me Thus, this indicates that a totally different class of cathepsin B inhibitor, a reversible inhibitor, is an effective AD drug because the data show that such an inhibitor reduces brain Aβ and brain (β-secretase activity. Reversible cathepsin B inhibitors effectively reverse the cognitive deficit caused by AD.

### Reversible Cathepsin B Inhibitor Reduces Brain Aβ & β-Secretase Activity in gp

Although the irreversible cathepsin inhibitors are efficacious, there is a potential concern that non-specific binding by these compounds may be the reason for their effect. Reversible inhibitors are thought to pose less of a concern with respect to specificity. In order to develop reversible cathepsin B inhibitors for AD, such compounds also need to be demonstrated to be efficacious. The peptidomimetic, acetyl-L-leucyl-L-valyl-L-lysinal (Ac-LVK-CHO) is a potent cell-permeable reversible cathepsin B inhibitor (4). Ac-LVK-CHO was administered to guinea pigs (gp) in the same manner as the irreversible inhibitors and brain Aβ and (β-secretase activity was determined.

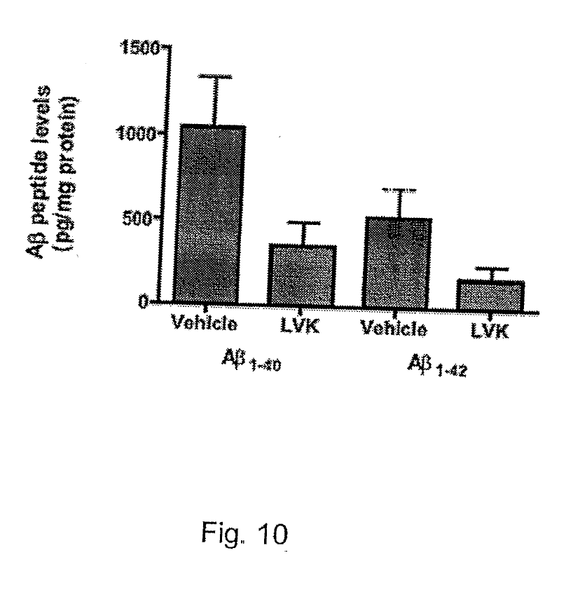

As shown in FIG. 10, Ac-LVK-CHO was icy infused into gp brains at 1 mg/ml, 2.5 μl/hr (0.15 mg/kg/day) for 30 days in a 1.5% DMSO/saline carrier solution using mini-pumps (N=8 animals per group). At the end of the treatment, the brains were isolated and extracted with guanidine hydrochloride and Aβ40 and Aβ42 levels were determined using ELISA. The controls were handled identically but did not receive Ac-LVK-CHO. The Aβ40 and Aβ42 levels for the treated brains and the controls are shown in FIG. 10 (mean+/−SD). Treating the animals significantly reduced both Aβ40 and Aβ42 by about 70% and 60%, respectively, relative to controls. These results were statistically significant with a p<0.0001.

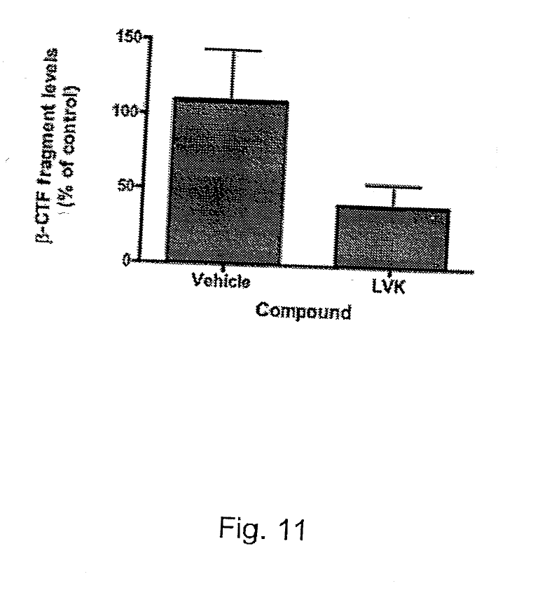

As shown in FIG. 11, Ac-LVK-CHO reduces gp brain CTFβ in vivo. Brains from animals described in relation to FIG. 10 were extracted and analyzed for CTFβ by ELISA. The CTFβ levels as a percent of control for the treated and control groups are shown in FIG. 11 (mean+/−SD). The treatment resulted in a about a 65% reduction in CTFβ. These results were statistically significant with a p<0.0001.

The reversible and irreversible inhibitors resulted in about the same reduction in brain Aβ and CTFβ in the gp model. Thus, a reversible inhibitor is likely to be as efficacious as an irreversible inhibitor, and the inhibition of cathepsin B is the most likely reason for this effect.

The data show that reversible cathepsin B inhibitors are also efficacious because a reversible inhibitor reduces brain Aβ and (β-secretase activity in vivo at about the same levels as did the efficacious irreversible inhibitors.

Peptide Structures Suitable for Drug Design have been Identified.

Reversible inhibitors suitable for drug development can be designed from peptide structures. As shown below, six dipeptide aldehydes having an IC₅₀ of 100 nM or less for inhibiting cathepsin B β-secretase activity are shown. These structures were found by screening a library of 70 dipeptide aldehydes. Cathepsin B β-secretase activity can be assayed using recombinant human cathepsin B cleavage of the peptidomimetic substrate, carbobenzoxy-Lvalyl-L-lysinal-L-methionyl-aminomethylcourmarinamide, Z-VKM-MCA. That substrate mimics the wild-type β-secretase cleavage site of human APP and its cleavage liberates a fluorescent MCA reporting molecule. Thus, peptide structures can be identified for design of reversible cathepsin B inhibitors suitable for drug development.

Peptide Structures Identified that Reversibly Inhibit Cathepsin B Activity

Peptide structures that inhibit cathepsin B β-secretase activity were used to develop small molecule AD drugs. A library of dipeptide aldehydes were screened for an inhibitory effect on the cathepsin B β-secretase activity using recombinant human cathepsin B (3.5 ng) cleavage of the Z-V-K-M-MCA substrate (100 μM). Over 70 dipeptide aldehydes were screened and 6 were found to have an IC₅₀ inhibitory concentration of 100 nM or less and are listed in Table 3

TABLE 3
Dipeptide Aldehydes Inhibitors of Cathepsin B β-Secretase Activity

| Formula | IC₅₀ inhibitory concentration (nM) |
|---|---|
| Z-VK(Ac)-ald | 31 |
| Z-YS-ald | 37 |
| Z-Q(Trt)S-ald | 58 |
| Z-YV-ald | 65 |
| Z-C(Z)S-ald | 67 |
| Z-C(Z)K(Ac)-ald | 91 |

### Design and Testing of Reversible Cathepsin B Inhibitors (Separate Out a Description of the Enzyme Inhibition Assay as a Separate Example)

The design of reversible small molecule cathepsin B inhibitors will be similar to that which has already been successfully employed to develop other cysteine proteases, such as IDN-6556, which is currently in phase II clinical trials for the treatment of liver disease. The discovery of IDN-6556 was accomplished through an iterative medicinal chemistry program starting from a known protease tetrapeptide inhibitor. The approach consisted of a number of steps, which are summarized here First, the tetrapeptide was truncated to reduce the peptide characteristics of the molecule. Reducing the peptide size to a tripeptide maintained the compound's potency but further truncation to a dipeptide abolished most of the inhibitory properties of the molecule Since it was desirable to build the pharmaceutical agent from a dipeptide core, a library of dipeptide aldehydes was prepared to investigate the effect of a variety of N-capping groups as potential surrogates for the P3 amino acid. This approach involved preparing a library of compounds utilizing solid phase parallel synthetic methodologies. This allowed for the synthesis of a large number of dipeptide aldehyde inhibitors with various N-capping groups. The capping groups were selected somewhat randomly with the intention of building a library that contained significant structural diversity. These investigations led to the identification a number of N-capped dipeptides which demonstrated excellent inhibitory activity against a broad spectrum of caspases. The final step leading to the identification of IDN-6556 was to investigate warheads other than the aldehyde group. This was due to the fact that the aldehyde warhead was not considered optimal for a pharmaceutical product and more importantly, the fact that the aldehydes, though potent against the isolated enzymes, were not adequately efficacious in whole-cell assays as inhibitors. Therefore, a study of various warheads was conducted and the resulting compound, IDN-6556, was identified as a clinical candidate Although the optimized IDN-6556 warhead is an irreversible warhead, many early compounds made in this process were reversible inhibitors.

This approach will also identify small molecule cathepsin B inhibitors suitable for drug development. In summary, the approach will be (1) identification of the optimal dipeptide core, (2) optimization of P3 interactions by random addition of various N-capping groups, and (3) replacement of the aldehyde warhead Similarly, molecular modifications of the peptide structures will result in reversible small molecule cathepsin B inhibitors suitable for further drug development

### Design of Reversible Small Molecule Cathepsin B Inhibitors

The approach to the discovery of reversible small molecule cathepsin B inhibitors follows the successful approach used in the identification of IDN-6556, discussed above. The steps involved in the medicinal chemistry development of IDN-6556 include: (1) identification of an optimal dipeptide core, (2) optimization of P3 interactions by random addition of various N-capping groups, and (3) identification of potential replacements for the aldehyde warhead.

As the first step, the known cathepsin B inhibitor, Ac-LVK-H, was truncated in an effort to define a dipeptide core from which to build the inhibitor. The structure of the final clinical candidate cannot be defined at this stage as the inhibitor will be developed during the iterative medicinal chemistry investigations. A library of dipeptide aldehydes consisting of all of the possible combinations of the twenty common amino acids was synthesized. For these initial test compounds, the N-terminus were capped with a carbobenzoxy (Z) group. The IC₅₀'s for the compounds against cathepsin B was determined. Several potent dipeptide aldehyde inhibitors have been identified, as shown in Table 3.

The second step in the development of the cathepsin B inhibitor was to enhance the potency of the dipeptide aldehydes by substituting the carbobenzoxy group at the N-terminus with a variety of alternate chemical groups. These groups were selected randomly with an emphasis on maximizing the diversity within this second library. The various groups were joined to the N-terminus via acylation or alkylation chemistry. The compounds were synthesized in parallel utilizing solid phase methodologies. It was anticipated that this phase would consist of several iterations, requiring the synthesis and testing of multiple sub-libraries of compounds. Several hundred compounds were synthesized and evaluated during this phase. The more potent analogs identified were tested in the vesicle assay. For those compounds where the structure activity relationship (SAR) against the isolated enzyme does not parallel the SAR of the analogs in the vesicle assay, further compounds were prepared as necessary in order to optimize the activity in the latter.

A third phase was the incorporation of alternate warheads. It is desirable to maintain the reversible inhibitory properties of the aldehydes and thus, warheads such as the nitrile warhead were synthesized and evaluated in the enzyme and vesicle assay. This third phase is optional as the aldehydes prepared in the second phase exhibit acceptable properties

### Synthesis of Reversible Small Molecule Cathepsin B Inhibitors.

The synthesis of a novel cathepsin B inhibitor follows the plan outlined in Scheme 1. The methodologies of scheme 1 are all documented in the literature. The first step is to convert the P1 amino acid to its aldehyde via conversion to the Weinreb amide followed by reduction with lithium aluminum hydride. Coupling of the aldehyde to a linker (structure and synthesis described in Scheme 2) via an imine functionality followed by attachment of the linker to a Wang resin provides the P1 aldehyde attached to a solid phase. Removal of the FMOC protecting group and standard amino acid coupling to the P2 residue provides the dipeptide aldehyde attached to a solid phase Removal of the FMOC followed by acylation or alkylation of the free amine with a variety of capping groups provides substituted dipeptide aldehydes. Final removal from the resin and purification gives rise to functionalized dipeptide aldehydes that can be evaluated for activity

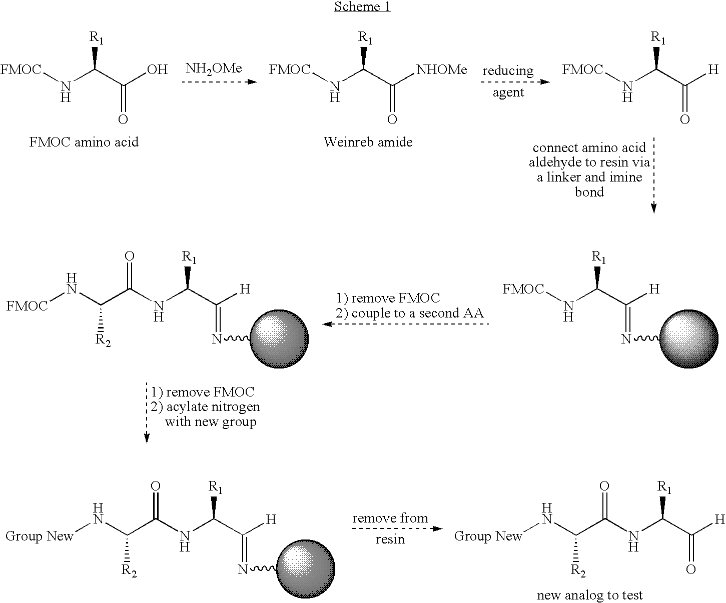

Synthesis of the known linker is outlined in Scheme 2. Tyramine is protected with a tBoc group and the hydroxyl group is alkylated with methyl bromoacetate Removal of the protecting group with acid followed by reaction with DCl and tBocNHNH₂ (tButylcarbazate) provides the fully protected linker, Saponification and final removal of the tBoc group provides the linker. The NH₂ group of the linker reacts with the amino acid aldehyde to form the imine. The carboxylic acid is attached to the amine on the resin via an amide bond.

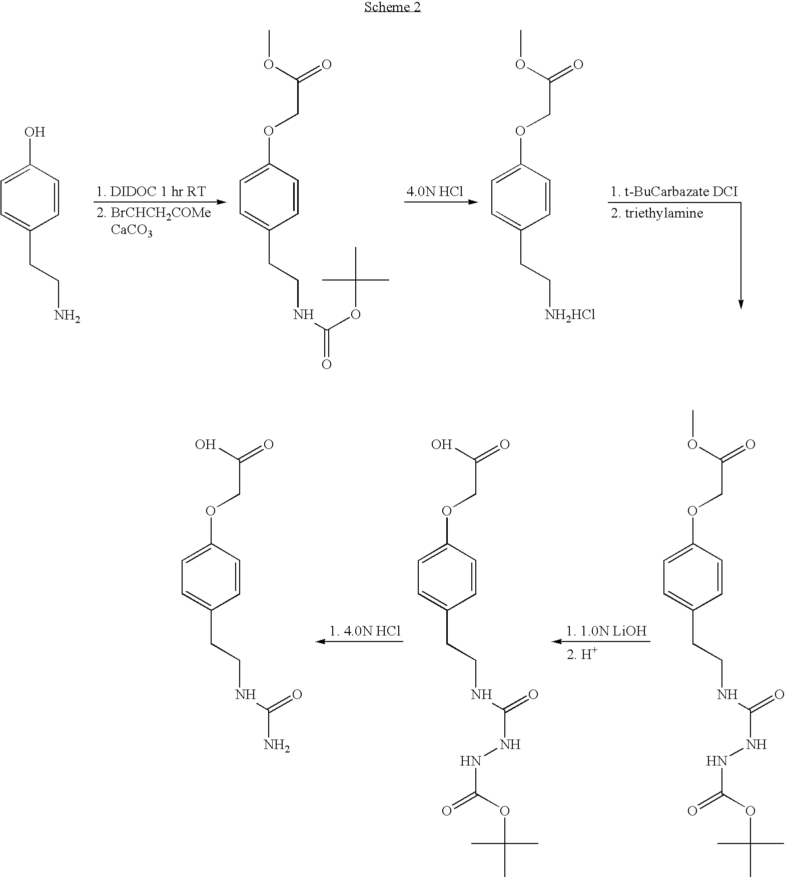

The small molecule cathepsin B inhibitors are selected for use in in vitro and in vivo assays that were used to select the irreversible inhibitors, loxistatin and CA074Me, and determine their efficacy. The in vitro assays include the cathepsin B β-secretase assay, the regulated secretory vesicle cathepsin B and Aβ assays, and regulated Aβ secretion cell assays. The in vivo assays use the APPIn mouse AD model and the guinea pig model of human APP processing

### Comparison of In Vivo Effects of Cathepsin B and BACE1 Inhibitors

The cathepsin B inhibitors, CA074Me, E64d and Ac-LVK-CHO, do not affect the aspartyl protease, BACE1. Thus, the reported data is not due to direct inhibition of BACE1.

As discussed above, removing cathepsin B in knock out animals does not appear to affect their viability, fertility, phenotype or behavior. Moreover, the cysteine protease inhibitor, E64d, has been shown to be safe in clinical trials. Although initially thought to be benign, recent data suggest that knocking out BACE1 may result in reduced viability and altered behavior (75). Thus, cathepsin B may be a safer target than BACE1 for inhibition.

As shown here, cathepsin B inhibitors improve cognitive function in an AD animal model whereas it appears that no BACE1 inhibitor has been shown to do this. Also, cathepsin B inhibitors reduce brain Aβ more than has been shown to occur for a BACE1 inhibitor. The effective dose of cathepsin B inhibitors is about 13,300 more potent than BACE1 inhibitors. Thus, the cathepsin B β-secretase inhibitors compare favorably to the BACE1 inhibitors in several respects.

### Other Cathepsin B Inhibitors and Cysteine Protease Inhibitors

In addition to the cathepsin B and cysteine protease inhibitors described above, any known cathepsin B inhibitor or cysteine protease inhibitor found suitable for pharmaceutical use can be used to treat or reverse the cognitive deficit caused by AD. For example, compounds described in International patent application no. WO 2007/038772, such as the prodrugs containing the brain targeted chemical delivery system coupled to a cysteine protease inhibitor moiety, compounds described in U.S. Patent Application Publication no 2004/0248232, and Frlan, R. and Gobec, S. (2006) Inhibitors of cathepsin B. Current Med. Chem. 13:2309-2327, are useful for this purpose. Others suitable inhibitors may include those described in U.S. Pat. Nos. 6,689,785, 6,387,908, 6,110,967, 5,843,992, 5,679,708, 5,556,853, 4,418,075 and 4,333,879. Assays described herein, in International patent application no. WO 2007/038772, and those known in the art can be used to select additional cathepsin B inhibitors or cysteine protease inhibitors for use in treating AD

### Pharmaceutical Use

The pharmaceutical compatibility of cathepsin B and cysteine protease inhibitors can be determined by known toxicological methods. As discussed above, neither inhibition of cathepsin B nor cysteine proteases are toxic. Suitable routes of administration can be readily determined based on animal studies. The effectiveness of the inhibitors for affecting AD can be assessed by any means known in the filed such as those described for AD at www.clinicaltrials.gov. For example, cognitive tests such as the Mattis Dementia Rating Scale and the MiniMental State Examination have been developed. Known neuroimaging methods can also be used for evaluating AD, especially Aβ, using various means such as magnetic resonance or positron emission tomography. Further, biomarkers associated with AD can be monitored for determining an effect, especially for levels of cathepsin B or cysteine protease activity in cerebral spinal fluid.

### EXPERIMENTAL METHODS

### In Vitro and Cell Assays

Novel in vitro screening assays that target β-secretase and Aβ production in the regulated secretory pathway of neuronal cells were used to identify cathepsin B inhibitors for (i) inhibition of β-secretase activity of recombinant cathepsin B, (ii) inhibition of endogenous β-secretase activity in regulated secretory vesicles, (iii) reduction of Aβ peptide production in regulated secretory vesicles, and (iv) reduction of Aβ secreted from the regulated secretory pathway from intact neuronal cells in primary culture. This series of unique assays identifies small molecules that effectively reduce Aβ peptide production in the major regulated secretory pathway of neuronal cells

### Recombinant Cathepsin B β-Secretase Assay

The recombinant cathepsin B β-secretase assay occurs in a solution (100 μl total volume) consisting of H₂O (20 μl), Na-Acetate (50 mM, pH 5.0), NaCl (100 mM), Z-VKM-MCA (100 μM), GSH (10 mM), EDTA (1 mM), and recombinant human cathepsin B (3.5 ng) and 2DMSO (2%). A dipeptide aldehyde was added to such solutions in varying amounts to determine the IC₅₀. The reagents were generally commercially available or, in the case of the dipeptide aldehydes, custom synthesized. The assay was incubated at 37° C. for 45 minutes and the fluorescence was read using a spectrophotometer (365/450 nm excitation/emission). Assays were run in duplicate. Compounds that inhibited cathepsin B by 80-100% (at 10 μM) underwent dose-response testing at 1 nm to 10 μM to determine potency, measured as IC₅₀ values (determined by non-linear regression a analysis using Graphpad Prism). Compounds with IC₅₀ values in the nanomolar range, up to 100 nM, were selected for further study.

### Dipeptide Aldehyde Library

A library of over 70 custom dipeptide aldehydes was screened in the cathepsin Bβ-secretase assay.

Inhibition of Endogenous β-Secretase Activity in Regulated Secretory Vesicles Isolated from Neuronal Chromaffin Cells

Cathepsin B compounds that effectively inhibit Cathepsin B in the Nanomolar range were tested for their abilities to inhibit endogenous β-secretase activity in regulated secretory vesicles isolated from neuronal chromaffin cells.

Isolation of regulated secretory vesicles, also known as chromaffin vesicles, from adrenal medullary tissue (bovine) was conducted by sucrose density gradient purification, as described previously (54-56, 58). Briefly, fresh bovine adrenal medulla (from 40 glands) was gently homogenized by a polytron (Brinkmann) in 200-250 ml ice-cold 0.32 M sucrose. The homogenate was centrifuged at 1500 rpm in a GSA rotor (Sorval centrifuge) for 20 minutes at 4° C. The supernatant was collected and centrifuged at 8000 rpm in a GSA rotor for 20 minutes at 4° C. to obtain a pellet of chromaffin vesicles. The pellet was washed 3 times in 0.32 M sucrose. After washing, the chromaffin vesicles were resuspended in 0.32 M sucrose and subjected to discontinuous sucrose gradient centrifugation in a SW28 rotor at 25,000 rpm for 120 min at 4° C. The pellets of isolated chromaffin vesicles were lysed in 0.015 M KCl with a glass-glass homogenizer and stored at −70° C. prior to use. Beta-secretase activities in frozen chromaffin vesicles are stable for at least 2 years

Endogenous β-secretase activity in lysed chromaffin vesicles was assayed with the β-secretase cleavage site specific substrate, Z-Val-Lys-Met-MCA, as described (38,39). Small molecules were first tested at a concentration of 10 μM; compounds that inhibit β-secretase activity by 80-100% were then analyzed using dose-response curves for measurement of IC₅₀ values. Compounds with IC₅₀ values in the nanomolar range (1-100 nM), were tested for inhibition of Aβ peptide production in chromaffin vesicles.

### Reduction of Aβ Peptide Production in Isolated Secretory Vesicles

Cathepsin B compounds that were effective inhibitors of endogenous II-secretase activity were tested for inhibition of Aβ peptide production in regulated secretory vesicles. The effect of reversible cathepsin B compounds on production of Aβ40 and Aβ42 in isolated chromaffin vesicles was tested by incubating candidate inhibitors (at 10 μM) with lysed chromaffin vesicles under identical conditions as for the β-secretase assay for 8 hours. This incubation resulted in NI peptide production in controls, which was inhibited by E64c or CA074Me (10 μM), as described previously (39). After incubation, Aβ40 and Aβ42 was measured by ELISA assays, as described for measurement of Aβ peptides in brain extracts. Each candidate inhibitor was assayed in triplicate. Cathepsin B compounds that showed at least 50% inhibition of Aβ production were tested in intact neuronal cells in primary culture for reduction of Aβ produced in the regulated secretory pathway.

Reduction of Aβ Secreted from the Regulated Secretory Pathway of Primary Neuronal Cell Cultures: Neuronal Chromaffin Cells and Brain Cortical Neuronal Cell Cultures

Reversible cathepsin B compounds that inhibited Aβ production in isolated secretory vesicles by at least 50% at 10 μM were tested for reduction of Aβ secreted from the regulated secretory pathway of primary neuronal chromaffin cells. Primary chromaffin cell cultures were prepared as described (50, 51). Cells were incubated with inhibitors (first at 50 μM) for about 18 hours, and then KCl-induced secretion of Aβ from the regulated secretory pathway was analyzed, by incubating cells in 50 mM KCl for about 15 minutes and measurement of Aβ peptides in the secretion medium Inhibitor effects were more readily detected during increased rates of Aβ production, which can be induced by treatment of cells with PMA (phorbol myristate acetate, 100 nM). Therefore, compounds were tested in the presence of PMA for effects on Aβ secreted from the regulated secretory pathway

Cathepsin B compounds that reduced regulated secretion of NI by at least about 50% were tested in brain cortical neurons in primary culture for effectiveness in brain neurons Guinea pig brain cortical neurons can be purchased, for example from GTS (Gene Therapy Systems, San Diego, Calif.), suitable for culturing. The tests for the ability of inhibitors to reduce Aβ secreted from the regulated secretory pathway of brain cortical neurons in primary culture were performed as described for chromaffin cells, with stimulation of regulated secretion by KCl depolarization. Compounds that effectively reduced regulated secretion of Aβ by at least about 50% were tested in vivo in guinea pigs and the London mutant mouse AD model

### Guinea Pig Assays

Intracerebroyentricular (Icv) Infusion into the Brain

Cathepsin B inhibitors were administered directly into the brain by icy infusion with minipumps in time course studies at different doses. Guinea pigs were infused with compounds using Alzet pumps (2.5 μl/hr, with compounds at 1 mg/ml in saline) for 7 and 30 days, and other times. This initial dose of compound was selected because E64d and CA074Me are effective at this dose. Each treatment group consisted of eight to ten guinea pigs, including the vehicle control group.

### Preparation of Brain Extracts and Synaptosomes for Aβ40 and Aβ42

### Measurements

### Brain Extracts and Synaptosome Preparations

At the end of each experiment, animals were sacrificed and half the brain was taken for ELISA measurement of Aβ40 and Aβ42 levels. The other half was isolated for synaptosome preparations for ELISA measurement of Aβ40 and Aβ42 content in nerve terminals. Synaptosomes were prepared by homogenization in isotonic sucrose (320 mM), pH 7.4, 1 mM EDTA, 0.25 mM dithiothreitol, and 30 U/MI RNAse inhibitor. The homogenate was centrifuged at 1000×g for 10 minutes. The supernatant was loaded on a Sucrose-Percoll discontinuous gradient and centrifuged at 32,000×g for 5 minutes (80) Enriched synaptosomes were washed in PBS, and centrifuged at 12,000×g for 4 minutes. The supernatant was removed, and the synaptosome containing pellet was resuspended in 50% OptiPrep (Accurate Chemica. Westbury, N.Y.), loaded on an OptiPrep discontinuous flotation gradient (9, 12.5, 25, and 35%) and centrifuged at 10,000×g for 20 minutes. The synaptosomes were obtained at the 15-25% interface

### Measurement of Aβ40 and Aβ42 Peptides

For quantitative analysis of Aβ peptides, an enzyme-linked immunosorbent assay (ELISA) was used to measure the levels of total Aβ, Aβₜₒₜₐₗ, Aβ40 and Aβ42 in the brains and synaptosomes of guinea pigs (Biosource International, Camarillo, Calif.) Aβₜₒₜₐₗ, Aβ40, Aβ42 was extracted from guinea pig brains or from synaptosomes by guanidine hydrochloride and quantified as described by the manufacturer. This assay extracted the total Aβ peptide from the brain (both soluble and aggregated). Tissue samples were homogenized in 5M guanidine buffer (5M guanidine HCl in 50 mM Tris-Cl, pH 8.3; Sigma-Aldrich, St. Louis, Mo.) at 1:10 w/v dilution. Homogenates were agitated at room temperature for 3-4 hours, then stored at −20° C. The day before running the ELISA, the tissue samples were diluted further 1:10 to provide a final guanidine concentration of 0.5M. These diluted homogenates were spun down at 14,000 rpm for 20 minutes at 4° C. and supernatants used for Aβ determination by ELISA (84-86). Brain and synaptosome extracts were assayed as diluted in 0.5M guanidine. In addition, Aβ40 and Aβ42 were assayed using ELISA using 6E10 as the capture antibody and Rb209 and Rb321 as reporter antibodies, respectively, for Aβ40 and Aβ42 (96).

### Transgenic APPIn Mice Assays

APPIn mice express the clinical mutant human APP (APP[V717I]) which has a mutation at the γ-secretase site that results in the overproduction of Aβ and the behavioral and neuropathology that develop an AD phenotype (8). These mice show early stage deficits in cognition and memory at 3-9 months age. At the later age of 8-12 months, amyloid plaques have developed in brains of these mice, with increased levels of Aβ40 and Aβ42 peptides in the brain. Early deficits in learning in these mice were reduced by a single administration of a AAV-CB-Aβ42 vaccine that induced in vivo production of anti-Aβ serum; vaccinated mice showed improved memory and cognition, decreased Aβ deposition, and decreased plaque development. Results shown here demonstrate that irreversible cathepsin B inhibitors reverse the cognitive deficits and neuropathology in the APPIn mouse model. Thus, this AD mouse model allows testing of select reversible compounds to improve cognition and to reduce brain levels of Aβ peptides and amyloid plaques.

The mouse model of AD expressing human APP containing the Swe mutant β-secretase site and the London mutation (APPswe/In) also overproduce Aβ, and develop neuropathology and behavior that mimic AD. APPswe/In was generated by site-directed mutagenesis at the β-secretase site of the APPIn to convert Lys-Met to the mutant Asn-Leu. Both APPswe/In and APPIn mouse strains were created in a C57BL/6 mouse background using the Thy-1.2 expression cassette driven by the Thy-1 promoter containing an SV40 polyadenylation site. The APPswe/In mice differ from the APPIn in that APPswe/In mice contain the Swedish mutant β-secretase site rather than the wt β-secretase site found in APPIn. Results shown here demonstrate that, as expected, irreversible cathepsin B inhibitors have no effect on APPswe/In mice

### Routes of Administration, Dose, and Schedule.

Direct icy infusion into the brain was the first route of administration to be tested to establish the effectiveness of reversible inhibitors in the brain to provide cognitive improvement, and reduction in brain Aβ peptides and amyloid plaques. Icy infusion was performed as described above for the irreversible inhibitors with use of the Alzet minipump. The initial dose utilized was the same dosage used for E64d and CA074Me in the APPIn mice or APPswe/In mice Administration of these compounds began at approximately 1-2 months of age and infusion was conducted for periods of 1, 2, 4, and 8 months (or other time periods). Administration of the select reversible compounds by direct icy infusion into mouse brains was conducted initially at 0.025 μl/hr of 1 mg/ml, other doses may be examined based on results with the first dose. Each time point was conducted with about 8-10 animals and 8-10 normal control animals (age-matched) (or greater number of mice per treatment group, if necessary, for appropriate statistical evaluation). At the end of each of these infusion periods, cognitive testing and assessment of Aβ peptide levels with plaque pathology was evaluated. At each of these time points, mice were tested for cognition, and then sacrificed for evaluation of Aβ and neuropathology in the brain

### Behavioral Testing for Cognitive Improvement: Morris Water Maze Test

The status of memory impairment in the APPIn or APPswe/In mice with and without administration of select compounds was assessed by the Morris water maze test. The water maze test is widely used in mouse AD animal models to evaluate cognitive impairment in such AD models. Water maze tests were conducted as previously described (87-89).

Drug-treated and vehicle-treated mice were tested in the Morris water maze test. Mice were trained in a 1.2 meter open field water maze. The pool was filled to a depth of 30 cm with water and maintained at 25° C. The escape platform (10 cm square) was placed 1 cm below the surface of the water. During the trials, the platform was removed from the pool. The cued test was carried out in the pool surrounded with white curtains to hide any extra-maze cues. Aβ animals underwent non-spatial pretraining (NSP) for three consecutive days. These trials were to prepare the animals for the final behavioral test to determine whether there was sufficient retention of memory to find the platform. These trials were not recorded since they were for training purposes only. For the training and learning studies, the curtains were removed to extra maze cues to allow identification of animals with swimming impairments. On day 1, the mice were placed on the hidden platform for 20 seconds (trial 1), for trials 2-3 animals were released in the water at a distance of 10 cm from the cued-platform or hidden platform (trial 4) and allowed to swim to the platform. On the second day of trails, the hidden platform was moved randomly between the center of the pool and the center of each quadrant. The animals were released into the pool, randomly facing the wall and were allowed 60 seconds to reach the platform (3 trials). In the third trial, animals were given three trials, two with a hidden platform and one with a cued platform. Two days following the NSP, the animals were subjected to final behavioral trials (Morris water maze test). For these trials (3 per animal), the platform was placed in the center of one quadrant of the pool and the animals were released facing the wall in a random fashion. Each animal was allowed to find the platform or swim for 60 seconds (latency period, the time it takes to find the platform). Aβ animals were tested within 4-6 hours of dosing and were randomly selected for testing by an operator blinded to the test group.

Statistical analyses utilized the StatView program, using student's t-test or by one-way ANOVA analyses for testing the significance of values. These analyses assessed changes in cognitive function with or without administration of the protease inhibitors.

### Aβ Peptide Levels and Amyloid Plaque Neuropathology in the Brain

After behavioral testing was completed, the mice were sacrificed for analyses of Aβ40 and Aβ42 peptide levels, and neuropathology. One half brain (one hemisphere) was utilized to prepare brain extracts for measurement of Aβ40 and Aβ42 levels by ELISA assays, and the other half brain was used for neuropathological evaluation. Procedures for preparation of brain extracts and ELISA assays for Aβ peptides are those used for analyses of guinea pig brains

Aβ-related neuropathology was assessed in cortex and hippocampus of brains. The relative extent of Aβ immunoreactivity in neurons and glial cells was assessed. Immunostaining for Aβ was conducted as described previously (58) Aβ-related neuropathology was assessed in cortex and hippocampus of brains, as well as other regions of the brain as controls. Brain tissue was fixed in 4% paraformaldehyde overnight, and then processed to paraffin. Serial sections were obtained, deparaffinized and washed in TBS buffer. Each section was blocked with appropriate normal serum (mouse or rabbit), and subjected to primary anti-Aβ antibody for detection of amyloid deposits. Sections were then stained with DAB (diaminobenzoic acid) with the Vector ABC Elite kit (Vector Laboratories). Amyloid areas were imaged with microscopic computer-assisted analysis system using the NIH Image Analysis Software.

### REFERENCES

1. Hook, V., Kindy, M., and Hook, G. (2006) Cysteine protease inhibitors effectively reduce in vivo levels of brain β-amyloid related to Alzheimer's disease. Biol. Chem., in press.
2. Miyahara, T., Shimojo, S., Toyohara, K., Imai, T., Miyajima, M., Honda, H., Kamegai, M., Ohzeki, M., and Kokatsu, J. (1985). Phase I study of EST, a new thiol protease inhibitor—the 2^{nd} report safety and pharmacokinetics in continuous administration. Rinsho Yakuri 16, 537-546.
3. Satoyashi, E. (1992). Therapeutic trials on progressive muscular dystrophy. Intern Med. 31, 841-846.
4. McConnell, R. M., York, J. L., Fizell, D., and Ezell, C (1993) Inhibition studies of some serine and thiol protineases by new leupeptin analogues. J. Med. Chem. 36, 1084-1089.
5. Beck, M., Muller, D., and Bigl, V. (1997). Amyloid precursor protein in guinea pigs—complete cDNA sequence and alternative splicing. Biochim. Biophys Acta 1351, 17-21
6. Johnstone, E. M., Chaney, M. O., Norris, F. H., Pascual, R., and Little, S. P. (1991) Conservation of the sequence of the Alzheimer's disease amyloid peptide in dog, polar bear and five other mammals. Mol. Brain. Res., 10, 299-305.
7. Beck, M., Bigl, V., and Robner, S. (2003) Guinea pigs as a nontransgenic model for APP processing in vitro and in vivo. Neurochem Res., 2003, 28, 637-644
8. Dewachter, I., Moechars, D., van Dorpe, J., Teseur, I., Van den Haute, C., Spittaels, K., and Van Leuven, F. (2001) Modelling Alzheimer's disease in multiple transgenic mice. Biochem. Soc. Symp., 67, 203-210
9. Standaert, D G and Young, A B (1996) Treatment of central nervous system degenerative disorders. In Goodman and Gilman's The Pharmacological Basis of Therapeutics (9^{th} edition), eds. J G Hardman, L E. Limbird, P B Milinoff, R W Ruddon, and A G Gilman. McGraw-Hill, Dallas, pp. 503-520
10. Iversen, L. L. Mortishire-Smith, R. J., Pollack, S. J., and Shearman, M. S. (1995) The toxicity in vitro of beta-amyloid protein. Biochem. J., 311, 1-16
11. Sisodia, S. S. (1999) Alzheimer's disease perspectives for the new millenium. J. Clin. Invest. 104, 1169-1170
12. Hardy, J and Selkoe, D J (2002) The amyloid hypothesis of Alzheimer's disease progress and problems on the road to therapeutics. Science, 2002, 297, 353-356
13. Calhoun, M E., Wiederhold, K-H., Abramowski, D., Phinney, A L., Probst, A., Sturchler-Pierrat, C., Staufenbiel, M., Sommer, B., and Jucker, M (1998) Neuron loss in APP transgenic mice. Nature, 395, 755-756
14 Games, D., Adams, D., Alessandrini, R., Barbour, R., Borthelette, P., Blackwell, C., Carr, T., Clemens, J., Donaldson, T., Gillespie, F., Guido, T., Hagopian, S., Johnson-Wood, K., Khan, K., Lee, M., Leibowitz, P., Lieberburg, I., Little, S., Masliah, E., McConlogue, L., Montoya-Zavala, M., Mucke, L., Paganini, L., Penniman, E., Power, M., Schenk, D., Seubert, P., Snyder, B., Soriano, F., Tan, H., Vitale, J Wadsworth, S., Wolozin, B., and Zhao, J. (1995) Alzheimer-type neuropathology in transgenic mice overexpressing V717β-amyloid protein. Nature, 373, 523-527
15. Higgins, L. S., Rodems, J. M., Catalano, R., Quon, D., and Cordell, B (1995) Early Alzheimer disease-like histopathology increases in frequency with age in mice transgenic for R-APP751 Proc. Natl. Acad. Sci. USA, 92, 4402-4406
16. Moechars, D., Lorent, K., De Strooper, B., Dewachter, I., and Van Leuven, F. (1996) Expression in brain of amyloid precursor protein mutated in the α-secretase site causes disturbed behavior, neuronal degeneration and premature death in transgenic mice. EMBO J., 15, 1265-1274
17. LaFerla, F. M., Tinkle, B. T., Bieberich, C. J., Haudenschild C. C., and Jay, G. (1995) The Alzheimer's Aβ peptide induces neurodegeneration and apoptotic cell death in transgenic mice. Nat. Genet., 9, 21-30
18 Borchelt, D. R., Thinakaran, G., Eckman, C. B., Lee, M. K., Davenport, F., Ratovitsky, T., Prada, C-M., Kim, G., Seekins, S., Yager, D., Slunt, H. H., Wang, R., Seeger, M., Levey, A. I., Gandy, S. E., Copeland, N. G., Jenkins, N. A., Price, D. L., Younkin, S. G., and Sisodia, S. S. (1996) Familial Alzheimer's disease-linked presenilin 1 variants elevate Aβ1-42/1-40 ratio in vitro and in vivo. Neuron, 17, 1005-1013.
19. Armogida, M., Petit, A., Vincent, B., Scarzello, S., Alves da Costa, C., and Checker, F. (2001) Endogenous β-amyloid production in presenilin-deficient embryonic mouse fibroblasts. Nat Cell Biol., 3, 1030-1033
20. Schenk, D., Barbour, R., Dunn, W., Gordon, G., Grajeda, H., Guido, T., Hu, K, Huang, J., Johnson-Wood, K., Khan, K., Kholodenko, D., Lee, M., Liao, Z., Lieberburg, I., Motter, R., Mutter, L., Soriano, F., Shopp, G., Vasquez, N., Vandevert, C., Walker, S., Wogulis, M., Yednock, T., Games, D. and Seubert, P. (1999) Immunization with amyloid-β attenuates Alzheimer-disease-like pathology in the PDAPP mouse. Nature, 400, 173-177
21. Younkin, S. G. (2001) Amyloid β vaccination: reduced plaques and improved cognition. Nat. Med., 7, 18-19.
22. Kang, J., Lemaire, H-G., Unterbeck, A., Salbaum, J. M., Masters, C. L., Grzeschik, K-H., Multhaup, G., Beyreuther, K., and Müller-Hill, B. (1987) The precursor of Alzheimer's disease amyloid A4 protein resembles a cell-surface receptor. Nature, 325, 733-736.
23. Tanzi, R. E., Gusella, J. F., Watkins, P. C., Bruns, G. A. P., St George-Hyslop, P., Van Keuren, M. L. Patterson, D., Pagan, S., Kurnit, D. M., and Neve, R. L. (1987) Amyloid β protein gene: cDNA, mRNA distribution, and genetic linkage near the Alzheimer locus. Science, 235, 880-884
24. Robakis, Ramakrishna, N., Wolfe, G., and Wisniewski, H. M. (1987) Molecular cloning and characterization of a cDNA encoding the cerebrovascular and the neuritic plaque amyloid peptide. Proc. Natl. Acad. Sci., USA, 84, 4190-4194,
25. Hook, V. Y. H., Eiden, L. E., and Brownstein, M. J. (1982) A carboxypeptidase processing enzyme for enkephalin precursors. Nature 295, 341-342.
26. Hook, V. Y. H. and Loh, Y. P. (1984) Carboxypeptidase B-like converting enzyme activity in secretory granules of rat pituitary. Proc. Natl. Acad. Sci. USA 81, 2776-2780.
27. Hook, V. Y. H., Azaryan, A. V., Hwang, S-R., and Tezapsidis, N. (1994) Proteases and the emerging role of protease inhibitors in prohormone processing. FASEB J. 8, 1269-1278.
28. Azaryan, A. V., Krieger, T. J., and Hook, V. Y. H. (1995) Purification and characteristics of the candidate prohormone processing proteases PC2 and PC 1/3 from bovine adrenal medulla chromaffin granules, J. Biol. Chem. 270, 8201-8208,
29. Schiller, M. R., Mende-Mueller, L., Moran, K., Meng, M., Miller, K. W., and Hook, V. Y. H. (1995) “Prohormone thiol protease” (PTP) processing of recombinant proenkephalin. Biochemistry 34, 7988-7995.
30, Yasothornsrikul, S., Aaron, W., Toneff, T., and Hook, V. Y. H. (1999) Evidence for the proenkephalin processing enzyme prohormone thiol protease (PTP) as a multicatalytic cysteine protease complex: activation by glutathione localized to secretory vesicles. Biochemistry 38, 7421-7430,
31. Hook, V. Y. H., Noctor, S., Sei, C. A., Toneff, T., Yasothornsrikul, S., and Kang, Y-H. (1999) Evidence for functional localization of the proenkephalin-processing enzyme, prohormone thiol protease, to secretory vesicles of chromaffin cells. Endocrinology 140, 3744-3754,
32. Taylor, C. V., Taupenot, L., Mahata, S. K., Mahata, M., Wu, H., Yasothornsrikul, S., Toneff, T., Caporale, C., Jiang, Q., Parmer, R. J. Hook, V. Y. H., and O'Connor, D. T. (2000) Formation of the catecholamine release-inhibitory peptide catestatin from chromogranin A Determination of proteolytic cleavage sites in hormone storage granules. J. Biol. Chem. 275, 22905-22915
33. Miller, R., Aaron, W., Toneff, T., Vishnuvardhan, D., Beinfeld, M., and Hook, V. Y. H. (2003) Obliteration of a-melanocyte-stimulating hormone derived from POMC in pituitary and brains of PC2-deficient mice. J. Neurochem. 86, 556-563
34. Yasothornsrikul, S., Greenbaum, D., Medzihradszky, K. F., Toneff, T., Bundey, R., Miller, R., Schilling, B., Petermann, I., Dehnert, J., Logvinova, A., Goldsmith, P., Neveu, J. M., Lane, W. S., Gibson, B., Reinheckel, T., Peters, C., Bogyo, M., and Hook, V. (2003) Cathepsin L in secretory vesicles functions as a prohormone-processing enzyme for production of the enkephalin peptide neurotransmitter. Proc. Natl. Acad. Sci. USA 100, 9590-9595
35. Hook, V., Yasothornsrikul, S., Greenbaum, D., Medzihradszky, K. F., Troutner, K., Toneff, T., Bundey, R., Reinheckel, T., Peters, C., and Bogyo, M. (2004) Cathepsin L and Arg/Lys aminopeptidase a distinct prohormone processing pathway for the biosynthesis of peptide neurotransmitters and hormones. Biol. Chem. 385, 473-480
36. Hook, V. Y. H. (2006) Protease pathways in peptide neurotransmission and neurodegenerative diseases. Cell. Mol. Neurobiol. in press
37. Hook, V. Y. H. (2006) Unique neuronal functions of cathepsin L and cathepsin B in secretory vesicles: biosynthesis of peptides in neurotransmission and neurodegenerative disease. Biol. Chem. in press
38. Zhou A, Webb G, Zhu X, Steiner D F. (1999) Proteolytic processing in the secretory pathway. J Biol Chem. 274, 20745-8
39. Seidah N G, Chretien M. (1997) Eukaryotic protein processing endoproteolysis of precursor proteins. Curr Opin Biotechnol. 8, 602-7
40. Fugere M, Day R. (2005) Cutting back on pro-protein convertases: the latest approaches to pharmacological inhibition. Trends Pharmacol Sci. 26, 294-301
41. Thomas G. (2002) Furin at the cutting edge from protein traffic to embryogenesis and disease. Nat Rev Mol Cell Biol 3, 753-66
42. Gainer H, Russell J T, Loh Y P. (1985) The enzymology and intracellular organization of peptide precursor processing: the secretory vesicle hypothesis Neuroendocrinology 40, 171-84
43. Docherty K, Steiner D F. (1982) Post-translational proteolysis in polypeptide hormone biosynthesis. Annu Rev Physiol 44, 625-38
44. Gumbiner, G., and Kelly, R. B. (1982) Two distinct intracellular pathways transport secretory and membrane glycoproteins to the surface of pituitary tumor cells. Cell 28, 51-55
45. Lodish, H., Berk, A., Zipursky, S. L., Matsudaira, P., Baltimore, D., and Darnell, J. (1999). Molecular Cell Biology, 4^{th} ed., W.H. Freeman, New York, pp. 691-726.
46. Nitsch, R. M., Slack, B. E., Wurtman, R. J., and Growdon, J. H. (1992). Release of Alzheimer's amyloid precursor derivatives by activation of muscarinic acetylcholine receptors. Science 258, 304-307.
47. Nitsch, R. J., Farber, S. A., Growdon, J. H., and Wurtman, R. J. (1993). Release of amyloid beta-protein precursor derivatives by electrical depolarization of rat hippocampal slices. Proc. Natl. Acad. Sci., USA 90, 5191-5193.
48. Farber, S. A., Nitsch, R. M., and Wurtman, R. J. (1995) Regulated secretion of beta amyloid precursor protein in rat brain. J. Neurosci. 15, 7442-7451
49. Cirrito, J. R., Yamade, K. A., Finn, M. B., Sloviter, R. S., Bales, K. R., May, P. C., Schoepp, D., Paul, S. M., Mennerick, S., and Holtzman, D. M. (2005) Synaptic activity regulates interstitial fluid amyloid-(3 levels in vivo. Neuron 48, 913-922
50. Hook, V. Y. H., Toneff, T., Aaron, W., Yasothornsrikul, S., Bundey, R., and Reisine, T. (2002) b-amyloid peptide in regulated secretory vesicles of chromaffin cells: evidence for multiple cysteine proteolytic activities in distinct pathways for b-secretase activity in chromaffin vesicles. J. Neurochem., 81, 237-256.
51. Hook, V., Toneff, T., Bogyo, M., Greenbaum, D., Medzihradszky, K. F., Neveu, J., Lane, Hook, G., and Reisine, T. (2005) Inhibition of cathepsin B reduces b-amyloid production in regulated secretory vesicles of neuronal chromaffin cells: evidence for cathepsin B as a candidate b-secretase of Alzheimer's disease. Biol. Chem., 386, 931-940.
52. Vassar, R., Bennet, B. D., Babu-Khan, S., Mendiaz, E. A., Denis, P., Teplow, D. B., Ross, S., Amarante, P., Loeloff, R., Luo, Y., Fisher, S., Fuller, J., Edenson, S., Lile, J., Jarosinski, M. A., Biere, A. L., Curran, E., Burgess, T., Louis, J. C., Collins, F., Treanor, J,. Rogers, G., and Citron, M. (1999) β-secretase cleavage of Alzheimer's amyloid precursor protein by the transmembrane aspartic protease BACE Science, 286, 735-741.
53. Yan, R., Bienkowski, M. J., Shuck, M. E., Miao, H., Tory, M. C., Pauley, A. M., Brashler, J. R., Stratman, N. C., Mathews, W. R., Buhl, A. E., Carter, D. B., Tomasselli, A. G., Parodi, L. A., Heinrikson, R L., and Gurney, M. E., (1999) Membrane-anchored aspartyl protease with Alzheimer's disease β-secretase activity. Nature, 402, 533-537.
54. Sinha, S., Anderson, J. P., Barbour, R., Basi, G. S., Caccavello, R., Davis, D., Doan, M., Dovey, H. F., Frigon, N., Hong, J., Jacobson-Croak, K., Jewett, N., Keim, P., Knops, J., Lieberburg, I., Power, M., Tan, H., Tatsuno, G., Tung, J., Schenk, D., Seubert, P., Suomensaari, S. M., Wang, S., Walker, D., Zhao, J., McConlogue, L., and John, V. (1999) Purification and cloning of amyloid precursor protein β-secretase from human brain. Nature, 402, 537-540.
55, Hussain, I, Powell, D, Howlett, D. R., Tew, D. G., Meek, T. D., Chapman, C., Gloger, I. S., Murphy, K. E., Southan, C. D., Ryan, D. M., Smith, T. S., Simmons, D. A., Walsh, F. S., Dingwall, C., and Christie, G. (1999) Identification of a novel aspartic protease (Asp 2) as (β-secretase. Mol. Cell. Neurosci., 14, 419-427.
56. Lin, Koelsch, G., Wu, S., Downs, D., Dashti, A., Tang, J. (2000). Human aspartic protease memapsin 2 cleaves the beta-secretase site of beta-amyloid precursor protein. Proc. Natl. Acad. Sci. USA 97, 1456-1460.
57. Mueller-Steiner, S., Zhou, Y., Arai, H., Robertson, E. D., Sun, B., Chen, J. Wang, X., Yu, G., Esposito, L., Mucke, L., and Gan, L. (2006) Antiamyloidogenic and neuroprotective functions of cathepsin B: implications for Alzheimer's disease Neuron 51, 703-714.
58. Takeda, M., Tanaka, S., Kido, H., Daikoku, S., Oka, M., Sakai, K., Katunuma, N. (1994) Chromaffin cells express Alzheimer's precursor protein in the same manner as brain cells. Neurosci. Lett. 168, 57-60.
59, Efthimiopoulos, S., Vassilacopoulou, D., Rippellino, J. A., Tezapsidis, N., and Robakis, N. K. (1996). Cholinergic agonists stimulate secretion of soluble full-length amyloid precursor protein in neuroendocrine cells. Proc. Natl. Acad. Sci, USA 93, 8046-8050.
60. Tezapsidis, N., Li, H. G., Rippellino, J. A., Efthimiopoulos, S., Vassilacopoulou, D., Sambamurti, K., Toneff, T., Yasothornsrikul, S., Hook, V. Y. H., and Robakis, N. K. (1998). Release of non-transmembrane, full-length Alzheimer's amyloid precursor protein (APP). from the lumenar surface of chromaffin granule membranes Biochemistry 37, 1274-1282,
61, Cataldo, A. M., and Nixon, R. A. (1990). Enzymatically active lysosomal proteases are associated with amyloid deposits in Alzheimer brain. Proc. Natl. Acad. Sci. USA 87, 3861-3865
62. Cataldo, A. M., Paskevich, P. A., Kominami, E., and Nixon, R A (1991) Lysosomal hydrolases of different classes are abnormally distributed in brains of patients with Alzheimer's disease, Proc Natl. Acad. Sci. USA 88, 10998-11002.
63. Zhang, J., Goodlett, D. R., Quinn, J., Peskind, E., Kaye, J. A., Zhou, Y., Pan, C., Yi, E., Eng., J., Wang, Q., Aebersold, R. H., and Montine, T. J. (2005) Quantitative proteomics of cerebrospinal fluid from patients with Alzheimer disease. J. Alzheimer's Disease 7, 125-133,
64. Bernstein, H. G., Kirschke, H., Wiederanders, B., Schmidt, D. and Rinne, A. (1990). Antigenic expression of cathepsin B in aged human brain. Brain Res. Bull. 24, 543-549.
65. Nakamura, Y., Takeda, M., Suzuki, H., Hattori, H., Tada, K., Hariguchi, S., Hashimoto, S, and Nishimura, T. (1991). Abnormal distribution of cathepsins in the brain of patients with Alzheimer's disease. Neurosci. Lett. 130, 195-198
66. Reinheckel, T., Deussing, T., Roth, W., Peters, C. (2001) Towards specific functions of lysosomal cysteine peptidases, phenotypes of mice deficient for cathepsin B or cathepsin L. Biol. Chem. 382, 735-741
67. Scrip (1992) 1765, 11
68. Setoyama, K., Koike, M., Abe, S., Tsutsui, Y., Tarumato, Y., and Nakane, S. (1986) Toxicological studies on ethyl(+)-(2S,3S)-3-[(S)-3-methyl-1-(3-methylbutylcabamoyl)butylcarbamoyl-2-oxiranecarboxylate (EST) acute toxicities of EST, its metabolite and a by-product. lyakuhin Kenkyu 17, 736-743.
69. Tamai, M., Matsumoto, K., Omura, S., Koyama, I., Ozawa, Y., and Hanada, K. (1986). In vitro and in vivo inhibition of cysteine proteinases by EST, a new analog of E-64. J. Pharmacobiodyn. 9, 672-677.
70. Towatari, T., Nikawa, T., Murata, M., Yokoo, C., Tamai, M., Hanada, K., and Katunuma, N. (1991). Novel epoxysuccinyl peptides, a selective inhibitor of cathepsin B in vivo. FEBS Lett. 280, 311-315.
71. Buttle, D. J., Murata, M., Knight, C. G., and Barrett, A. J. (1992) CA074 methyl ester: a proinhibitor for intracellular cathepsin B. Arch. Biochem. Biophys., 299, 377-380.
72. Gruninger-Leitch, F., Schlatter, D., Kung, E., Nelbock, P., and Dobeli, H. (2002) Substrate and inhibitor profile of BACE (beta-secretase) and comparison with other mammalian aspartic proteases. J. Biol. Chem. 277, 4687-4693
73. Roberds, S. L., Anderson, J., Basi, G., Bienkowski, M. J., Branstetter, D. G., Chen, K. S., Freedman, S. B., Frigon, N. L., Games, D., Hu, K., Johnson-Wood, K., Kappenman, K. E., Kawabe, T. T., Kola, I., Kuehn, R., Lee, M., Liu, W., Motter, R., Power, M., Robertson, D. W., Schenk, D., Schoor, M., Shopp, G. M., Shuck, M E., Sinha, S., Svensson, K. A., Tatsuno, G., Tintrup, H., Wijsman, J., Wright, S., McConlogue, L. (2001). BACE knockout mice are healthy despite lacking the primary beta-secretase activity in the brain implications for Alzheimer's disease therapeutics, Hum. Mol. Genet. 10, 1317-1324.
74, Cai, Wang, Y., McCarthy, D., Wen, H., Corchelt, D. R., Price, D. L., and Wong, P. C. (2001), BACE 1 is the major β-secretase for generation of Aβ by neurons. Nat. Neurosci. 4, 233-234
75. Dominguez, D., Tournoy, J., Hartmann, D., Huth, T., Cryns, K., Deforce, S., Serneels, L., Camacho, I. E., Marjaux, E., Craessaerts, K., Roebroek, A. J. M., Schwake, M., D'Hooge, R., Bach, P., Kalinke, U., Moechars, D., Alzheimer, C., Reiss, K., Saftig, P., and De Strooper, B. (2005) Phenotypic and biochemical analysis of BACE1- and BACE2-deficient mice. J. Biol. Chem. 35, 30797-30806
76. Luo, Y., Bolon, B., Kahn, S., Bennet, B. D., Babu-Khan, S., Denis, P., Fan, W., Kha, H., Ahang, J., Gong, Y., Martin, L., Louis, J. C., Yan, Q., Richards, W. G., Citron, M., Vassar, R. (2001). Mice deficient in BACE 1, the Alzheimer's β-secretase, have normal phenotype and abolished β-amyloid generation. Nat. Neurosci. 4, 231-232
77. Laird, F. M., Cai, H., Savonenko, A. V., Farah, M. H., He, K., Melnikova, T., Wen, H., Chiang, H. C., Xu, G., Koliatsos, V. E., Borchelt, D. R., Price, D. L., Lee, H. K., Wong, P. C. (2005) BACE1, a major determinant of selective vulnerability of the brain to amyloid-beta amyloidogenesis, is essential for cognitive, emotional, and synaptic functions. J. Neurosci. 25, 11693-11709
78. Chang, W. P., Koelsch, G., Wong, S., Downs, D., Da, H., Weerasena, V., Gordon, B., Devasamudram, T., Bilcer, G., Ghosh, A. K., and Tang, J. (2004) In vivo inhibition of Abeta production by memapsin 2 (beta-secretase) inhibitors J. Neurochem. 89, 1409-1416
79. Asai, M., Hattori, C., Iwata, N., Saido, T. C., Sasagawa, N., Szabo, B., Hashimoto, Y., Maruyama, K., Tanuma, S., Kiso, Y., Ishiura, S (2006) The novel beta-secretase inhibitor KMI-429 reduces amyloid beta peptide production in amyloid precursor protein transgenic and wild-type mice. J, Neurochem. 96, 533-540
80. Dunkley, P. R., Jarvie, P. E., Heath, J. W., Kidd, G J., and Rostas, J. A. P. (1986) A rapid method for isolation of synaptosomes on Percoll gradients, Brain Res., 372, 115-129
81. Frlan, R. and Gobec, S. (2006) Inhibitors of cathepsin B. Current Med Chem. 13:2309-2327.

Each of the references cited within is expressly incorporated herein in its entirety. Although the invention has been described with references to the examples provided above, modifications can be made without departing from the invention. Accordingly, the invention is limited only by the claims.

1. A method of treating Alzheimer's disease comprising the step of administering an effective amount of an inhibitor of a cysteine protease to a patient in need thereof and thereby causing a reversal in the progression of the Alzheimer's disease and/or an improvement in a cognitive deficit.

2. The method of claim 1, wherein the cysteine protease inhibitor comprises a cathepsin B inhibitor.

3. The method of claim 1, wherein the reversal in the progression of the Alzheimer's disease comprises an improvement in cognitive deficit.

4. The method of claim 1, wherein administration of the cathepsin B inhibitor causes a reduction in Aβ and/or amyloid plaque.

5. The method of claim 2, wherein the cathepsin B inhibitor comprises an E64c or an E64d.

6-7. (canceled)

8. A method of treating an individual having a cognitive deficit comprising administering an effective amount of one or more cathepsin B inhibitors to an individual in need thereof, thereby causing an improvement in the cognitive deficit and a reduction in Aβ and/or amyloid plaque.

9. The method of claim 1, wherein the E64d is administered orally.

10. The method of claim 1, wherein the E64c or E64d is administered at a dosage of approximately 5 mg/kg/day.

11. The method of claim 8, wherein the cathepsin B inhibitor comprises E64c or E64d.

12. The method of claim 11, wherein the E64d is administered orally.

13. The method of claim 11, wherein the E64c or E64d is administered at a dosage of approximately 5 mg/kg/day.

14. A method of treating Alzheimer's disease or a cognitive deficit comprising the step of administering an effective amount of an E64c or an E64d to a patient in need thereof and thereby causing a reversal in the progression of the Alzheimer's disease and/or an improvement in a cognitive deficit.

15. The method of claim 14, wherein the E64d is administered orally.

16. The method of claim 15, wherein the E64c or E64d is administered at a dosage of approximately 5 mg/kg/day.
